(19) United States
(12) Patent Application Publication
(10) Pub. No.: US 2024/0293326 A1
(43) Pub. Date: Sep. 5, 2024

(54) CONTROLLED RELEASE FORMULATIONS FOR THE TREATMENT OF MALARIA

(71) Applicant: KARICI DIAGNOSTICS INC., Dorval (CA)

(72) Inventors: Mircea-Alexandru MATEESCU, Montreal (CA); Kouadio Victorien KONAN, Montreal (CA)

(21) Appl. No.: 18/612,370

(22) Filed: Mar. 21, 2024

Related U.S. Application Data
(63) Continuation of application No. 17/266,809, filed on Feb. 8, 2021, now abandoned.
(60) Provisional application No. 62/739,394, filed on Oct. 1, 2018.
(60) Provisional application No. 62/715,992, filed on Aug. 8, 2018.

(51) Int. Cl. A61K 9/20; A61K 31/357; A61K 36/24

(52) U.S. Cl. CPC A61K 9/2054; A61K 9/2059; A61K 9/2086; A61K 31/357; A61K 36/24

(57) ABSTRACT

The present disclosure relates to a controlled release complex of a carboxylated polymer having carboxyl groups having a degree of substitution of about 0.1 to about 1.0, forming a complex through ionic interaction with an antimalarial alkaloid extract; a solid oral dosage form comprising the same, and a solid oral dosage form comprising an antimalarial drug combination which comprises the controlled release complex of the present invention and an antimalarial drug.

### CROSS-REFERENCE TO RELATED APPLICATIONS

This application is a continuation of U.S. patent application Ser. No. 17/266,809 filed Feb. 8, 2021, which is a U.S. National Phase Application under 35 U.S.C. § 371 of PCT/CA2019/051057, filed Aug. 1, 2019, which claims priority from and the benefit of U.S. Provisional Patent Application No. 62/715,992 filed on Aug. 8, 2018 and of U.S. Provisional Patent Application No. 62/739,394 filed on Oct. 1, 2018, the specifications of which are hereby incorporated by reference in their entireties.

### BACKGROUND

### (a) Field

The subject matter disclosed generally relates to control release complexes, and more specifically to control release complexes of carboxylated polymers with antimalarial alkaloid extract. The subject matter disclosed also relates to oral dosage forms comprising the control release complex alone or with other antimalarial drugs. The subject matter disclosed also relates to methods of preparing the control release complexes of the present invention.

### (b) Related Prior Art

Malaria parasite, Plasmodium, infects hundreds of millions of people yearly and causes hundreds of thousands of deaths in various endemic areas, mainly in tropical and subtropical areas endemic disease. Although to a lesser extent, the Middle East and parts of Eastern Europe are also affected by this endemic disease. About half of the world's population is exposed to the female anophele mosquito which is the principal vector of Plasmodium falciparum, responsible for over 80% of infections and about 90% of deaths from malaria.

Malaria therefore remains a major public health problem with hundreds of thousands deaths yearly, mostly children. Although Artemisinin is still considered effective, in order to avoid the appearance of new resistant strains, the World Health Organization (WHO) recommends Artemisinin Combination Therapy (ACT) associated to a bioactive agent with a long half-life and/or a sustained release formulation ensuring a long duration of antiplasmodial activity. Antimalarial alkaloid extracts such as Peschiera fuchsiaefolia(Pf) alkaloid are good candidates for ACT, but the solubility of Pf is very low. Previous studies proposed to ameliorate the solubility of Pf by coating with maltodextrin but the product is unstable.

Therefore, there is a need for new solutions to improve Pf solubility.

### SUMMARY

According to an embodiment, there is provided a controlled release complex comprising:
a carboxylated polymer having carboxyl groups having a degree of substitution of about 0.1 to about 1.0, forming a complex through ionic interaction with an antimalarial alkaloid extract, wherein the carboxylated polymer and the antimalarial alkaloid extract are present in a ratio of from about 10:90 to about 1:99.

The carboxylated polymer may be selected from the group consisting of carboxymethylcellulose (CMC), carboxymethylstarch (CMS), or combinations thereof.

The carboxymethylcellulose may have a degree of substitution of about 0.7 to about 0.8. The carboxymethylcellulose may have a degree of substitution of about 0.8. The carboxymethylstarch may have a degree of substitution of about 0.1 to about 0.3. The carboxymethylstarch may have a degree of substitution of about 0.27.

The carboxylated polymer and the antimalarial alkaloid extract may be present in a ratio of about 4:96.

The antimalarial alkaloid extract may be selected from the group consisting of an alkaloid extract from Guiera senegalensis, Strychnos usambarensis, Balanites rotundifolia and Peschiera fuchsiaefolia and preferably Peschiera fuchsiaefolia.

According to another embodiment, there is provided a solid oral dosage form comprising the controlled release complex of the present invention and pharmaceutically acceptable excipients.

The solid oral dosage form may further comprise a binding agent.

The binding agent may be selected from the group consisting of hydroxypropyl methylcellulose (HPMC), sucrose, lactose, starches, cellulose, microcrystalline cellulose, xylitol, sorbitol, mannitol, gelatin, hydroxypropyl cellulose (HPC), polyvinylpyrrolidone (PVP), and polyethylene glycol (PEG).

The solid oral dosage form may be further comprising a lubricant.

The lubricant may be selected from the group consisting of magnesium stearate, talc, silica, vegetable stearin, and stearic acid.

The dosage form may be monolithic.

According to another embodiment, there is provided a solid oral dosage form for the controlled release of an antimalarial drug combination, comprising:
the controlled release complex of the present invention;
an antimalarial drug, and
pharmaceutically acceptable excipients.

The antimalarial drug may be selected from the group consisting of quinine, artemisinin, artesunate, artemether, arteether, dihydroartemisinin, and artelinate. The antimalarial drug may be artemisinin.

The solid oral dosage form may be further comprising a binding agent.

The binding agent may be hydroxypropyl methylcellulose.

The solid oral dosage form may be further comprising a lubricant.

The lubricant may be magnesium stearate.

The solid oral dosage form may be further comprising an additional carboxylated polymer, uncomplexed with the antimalarial alkaloid extract.

The solid dosage form may be monolithic or multilayered.

The solid oral dosage form may be a bilayer.

According to another embodiment, there is provided a method of treating

malaria comprising administering to a subject in need thereof an effective amount of the solid dosage form of the present invention.

According to another embodiment, there is provided a use of the solid dosage form of the present invention for the treatment of malaria in a subject in need thereof.

According to another embodiment, there is provided a solid oral dosage form of the present invention for the treatment of malaria in a subject in need thereof.

The use of the controlled release complex of the present invention may be for the manufacture of a medicament for treatment of malaria in a subject.

The use of the solid oral dosage form of the present invention may be for the manufacture of a medicament for treatment of malaria in a subject.

According to another embodiment, there is provided a process for the preparation of a controlled release complex of an antimalarial alkaloid extract comprising the steps of:
contacting a carboxylated polymer having carboxyl groups and having a degree of substitution of about 0.1 to about 1.0 suspended in water with the antimalarial alkaloid extract dissolved in a solvent-water medium.

The solvent-water medium may be a hydroalcoholic medium.

The hydroalcoholic medium may be 60% v/v ethanol in water.

The carboxylated polymer may be selected from the group consisting of carboxymethylcellulose (CMC), carboxymethylstarch (CMS), or combinations thereof.

The carboxymethylcellulose may have a degree of substitution of about 0.7 to about 0.8. The carboxymethylcellulose may have a degree of substitution of about 0.8. The carboxymethylstarch may have a degree of substitution of about 0.1 to about 0.3. The carboxymethylstarch may have a degree of substitution of about 0.27.

The carboxylated polymer and the antimalarial alkaloid extract are present in a ratio of about 4:96.

The antimalarial alkaloid extract may be selected from the group consisting of an alkaloid extract from Guiera senegalensis, Strychnos usambarensis, Balanites rotundifolia and Peschiera fuchsiaefolia and preferably Peschiera fuchsiaefolia.

The following terms are defined below.

As used herein, the term «functionalizing starch» or «functionalized starch» or «functionalizing cellulose» or «functionalized cellulose» is intended to mean functionalization that is not limited to the conversion of the native or modified starch or cellulose by carboxymethylation, but also includes possible functionalization of other starch derivatives such as starch succinate (succinyl starch), hydroxypropyl starch, acetyl starch, hydroxypropyl methyl starch, acid modified starch, octenyl starch, pregelatinized starch or mixture thereof.

The term «functionalization» as used herein is intended to mean the addition by covalent bonds of carboxyl groups (or its derivatives) onto the starch or cellulose chains. The functionalization can be (but is not limited to) the carboxylation (addition of carboxylate groups), amination (addition of amine groups), alkylation (addition of alkyl groups) or acylation (addition of acyl groups).

The terms «carboxylation» >and «carboxylated» as used herein are intended to mean the addition of carboxyl groups onto the polysaccharide macromolecule. Possible carboxylation includes but not limited to the carboxymethylation, carboxyethylation, succinylation, acrylation, etc. According to a preferred embodiment, the carboxylation is a «carboxymethylation».

The term «degree of substitution» is intended to mean the average number of substituents per glucose unit (GU), the monomer unit of starch. Since each GU contains three hydroxyl groups, the DS can vary between 0-3, and may be expressed also in percentage. According to an embodiment of the present invention, the DS may be equal to or greater than 0.2 such as to obtain for certain BA up to 1.0, and in certain preferred embodiment ≥0.6 incorporated in the functionalized carboxyl polymer (e.g. CMS).

The terms «complexation» and «complex» are intended to mean the process by which the carboxylated polymer having carboxyl groups is mixed with the antimalarial alkaloid extract. The complexation reaction with the carboxyl groups and antimalarial alkaloid extract is performed by mixing suspensions of the carboxylated polymer having carboxyl groups in water in the antimalarial alkaloid extract in alcohol, thereby forming a complex. The formed complex is spray dried with gentle stirring to prevent sedimentation and obtain the complex in powder form. According to an embodiment, the complex is formed by the ionic interaction of the antimalarial alkaloid extract with the carboxyl group of the carboxylated polymer.

The term «composition » as used herein is intended to encompass a product comprising the specified ingredients in the specified amounts, as well as any product which results, directly or indirectly, from combination of the specified ingredients in the specified amounts. Such term in relation to pharmaceutical composition or other compositions in general, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions or other compositions in general of the present invention encompass any composition made by admixing a compound of the present invention and a pharmaceutically acceptable carrier. By “pharmaceutically acceptable” or “acceptable” it is meant the carrier, diluent or excipient must be compatible with the other ingredients of the formulation and not deleterious to the recipient thereof.

Before describing the present invention in detail, a number of terms will be defined. As used herein, the singular forms “a”, “an”, and “the” include plural referents unless the context clearly dictates otherwise.

It is noted that terms like “preferably”, “commonly”, “such as” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates the chemical structures of alkaloids from Pf extract (from Ramanitrahasimbola et al., 2001);

FIG. 2 illustrates the complexation of Pf to CMS according to DS of the CMS;

FIG. 3 illustrates the FTIR spectra of native starch and different Carboxymethylstarch (CMS) derivatives;

FIG. 4 illustrates the FTIR spectra of MD, Pf free, CMS, MD/Pf and CMS-Pf complex;

FIG. 5 illustrates the FTIR spectra of CMS-Pf and MD/Pf after incubation in simulated gastric (SGF) and intestinal (SIF) fluids;

FIG. 6 illustrates X-Ray diffractograms of CMS with different DS;

FIG. 7 illustrates X-Ray diffractograms of MD, MD/Pf coated, CMS and CMS-Pf complexes. MD/Pf and CMS-Pf in SGF or in SIF are in tablet dry forms. These samples were obtained after incubation of tablet forms of MD/Pf or CMS-Pf in SGF or in SIF and then dried before X-ray analysis.

FIG. 8 illustrates Scanning Electronic Microscopy (SEM) of Pf, MD/Pf coating and CMS-Pf complexes;

FIG. 9 illustrates the antioxidant capacity of Pf, MD/Pf and CMS-Pf in water, SGF and SIF before (Oh) and after incubation (2h) in each medium;

FIG. 10 illustrates the solubility in saturation condition of Pf, MD/Pf and CMS-Pf in different media using drying method;

FIG. 11 illustrates the solubility of Pf, MD/Pf and CMS-Pf using the new approach based on the absorption capacity of Pf at 280 nm and on its antioxidant capacity in different media at various times of incubation : 5 h (a) and 24 h (b). Solubilities were quantified by spectrophotometric measurement at 280 nm and by antioxidant capacity of Pf. No significant difference was obtained.

FIG. 12 illustrates the release profiles (A-C) and release time (D) of Peschiera extracts from tablets of 755-855 mg formulated with CMS as matrix containing 450 mg Pf as API, magnesium stearate (lubricating agent) and a binding agent (HPMC). Tablets also contained CMS1 or CMS2 or CMS3 (120 mg), with different amounts of HPMC in formulation F1 (100 mg), F2 (120 mg), F3 (150 mg), F4 (200 mg) and 15 mg magnesium stearate. The four formulations differed in terms of CMS (CMS1, CMS2 or CMS3) HPMC amounts quantity: F1, F2, F3 and F4 were formulated with 100 mg, 120 mg, 150 and 200 mg of HPMC. When the matrix used was CMS1, the formulations were labeled as F1.1, F2.1, F3.1 and F4.1. These release profiles represent quantities of released Pf in 1000 mL of simulated gastric fluid (SGF, pH 1.5) during 2 h and then in 1000 mL of simulated intestinal fluid (SIF, pH 6.8), at 37° C. and 100 rpm, followed with a dissolution device Distek (Apparatus 2);

FIG. 13 illustrates the color intensities and absorbencies at 280 nm (λmax) of Pf, MD/Pf and CMS-Pf saturated solutions in different media. Values on pictures are OD_{280 nm} obtained after dilution (1/500) of samples;

FIG. 14 illustrates the solubility of free Pf and CMC/Pf complex in SGF;

FIG. 15 illustrates X-Ray diffractograms of CMS and of CMC complexes with Pf;

FIG. 16 illustrates FTIR spectra of CMS, CMC and their complexes with Pf.

FIG. 17 illustrates the release kinetics of artemisinin/Pf combination formulated as bilayer tablet dosage form according to an embodiment of the present invention;

FIG. 18 illustrates FTIR spectra of maltodextrin (MD) and carboxymethylcellulose (CMC) free and complexed with Peschiera fuchsiaefolia (Pf).

FIG. 19 illustrates FTIR spectra of maltodextrin (MD) and carboxymethylcellulose (CMC) complexed with Pf after incubation in simulated gastric (SGF) and simulated intestinal (SIF) fluids.

FIG. 20 illustrates a scanning electron microscopy (SEM) of MD/Pf and CMC/Pf aggregates;

FIG. 21 illustrates a SEM schematical representation and hypothetical structure of MD aggregated with Pf;

FIG. 22 illustrates the release kinetic profile of artemisinin combined with Pf, in a monolithic dosage form.

FIG. 23 illustrates the dosage methods of: A) Pf (i.e voacamin) by UV Spectrophotometric method; B) Arte: Scission of Artemisinin endoperoxide bond generating hydrogen peroxide which may react with ABTS to produce ABTS^{•+} (colored product, A_{734 nm}).

FIG. 24 illustrates FT-IR spectra of: A) MD and CMC free and complexed with Pf; B) MD/Pf and CMC-Pf after incubation in simulated gastric fluid (SGF) and then in simulated intestinal fluid (SIF).

FIG. 25 illustrates X-ray diffractograms of MD, CMC and of coated MD/Pf coating and CMC-Pf complexes before and after incubation in SGF and in SIF.

FIG. 26 illustrates scanning Electron Microscopy of MD/Pf aggregates and of CMC-Pf complexes according to embodiments of the present invention.

FIG. 27 illustrates thermograms of A) maltodextrin (MD) and coated MD/Pf; B) CMC and CMC-Pf complex according to embodiments of the present invention.

FIG. 28 illustrates the antioxidant capacity of MD/Pf and CMC-Pf in nanopure water, SGF and SIF.

FIG. 29 illustrates the solubility (mg/ml) of MD/Pf aggregate and of CMC-Pf complex in nanopure water, SGF and SIF.

FIG. 30 illustrates quantification methods where A) UV-Vis absorption spectrum of CMC-Pf complex; B-D) Standard curve of Pf by Spectrophotometric at 280 nm; E) Standard curve of Arte by ABTS reagent.

FIG. 31 illustrates the behavior of Arte (or CMC-Pf) in the presence of CMC-Pf (or Arte) after incubation first in SGF and then in SIF, one in the presence or no of the other: A) FTIR spectra of Arte; B) FTIR spectra of CMC-Pf; C) 1HNMRspectra of Arte and D) Antioxidant capacity of CMC-Pf in the presence or not of the Artemisinin.

FIG. 32 shows pictures of bilayer (A) and monolithic (B) tablets for ACT with Arte and CMC-Pf and (C) size of tablets (length, width and thickness).

FIG. 33 illustrates the kinetic release profiles of combined Artemisinin and CMC-Pf tablet under: A) bilayer and B) monolithic dosage forms.

FIG. 34 illustrates different quantities (200-400 mg) of CMC-Pf complex powder (without excipient) in the die.

FIG. 35 illustrates the influence of the precompression forces on volume (A) and tap density (B) of precompressed CMC-Pf powders according to embodiments of the present invention. The initial volume of uncompressed CMC-Pf powder was considered as 100%.

FIG. 36 illustrates the influence of tapping time on volumes of pCMC-Pf precompressed at different forces (25, 50 and 100 Kg/g).

FIG. 37 illustrates FTIR of CMC-Pf and pCMC-Pf at different precompression forces.

FIG. 38 illustrates X-RD diffractograms of CMC-Pf and of pCMC-Pf complexes precompressed at various forces.

FIG. 39 illustrates TGA diagrams and TG parameters of CMC-Pf and of pCMC-Pf submitted at different precompression forces.

FIG. 40 illustrates SEM micrographs of CMC-Pf and of pCMC-Pf (CMC-Pf precompressed at 25 Kg/g).

FIG. 41 illustrates the solubility (A) and antioxidant activity (B) of CMC-Pf and of pCMC-Pf precompressed at various forces in aqueous media, simulated gastric (SGF) and intestinal (SIF) fluids.

FIG. 42 illustrates release profiles of CMC-Pf, pCMC-Pf and Arte from ACT monolithic tablet dosage forms as bitherapeutic agents.

FIG. 43 illustrates the mechanical properties: A) Friability and B) Hardness determined taking the length (I) or the width (w) of the tablet as diameter (dₗ and d_{w}). Different tablets were obtained using (as active ingredient) the pCMC-Pf precompressed at various forces (0, 25, 50 and 100 Kg/g).

It will be noted that throughout the appended drawings, like features are identified by like reference numerals.

### DETAILED DESCRIPTION

In embodiments there is disclosed a controlled release complex comprising:
a carboxylated polymer having carboxyl groups having a degree of substitution of about 0.1 to about 1.0, forming a complex through ionic interaction with an antimalarial alkaloid extract, wherein the carboxylated polymer and the antimalarial alkaloid extract are present in a ratio of from about 10:90 to about 1:99.

Indole alkaloids represent an interesting class of natural compounds. Quinine (a quinoline alkaloid) and its derivatives have been, for many years, as principal treatment of malaria. This monotherapy was abandoned due to the diminution of sensibility of Quinine and to prevent the appearance of new resistant strains, particularly of Plasmodium falciparum. Infections caused by these parasitic protozoa take an enormous toll on humans, particularly in tropical and sub-tropical countries. Malaria is considered as the first parasitic disease with about 230,000 deaths in 2016 in 91 countries. Artemisinin (Arte) is also efficient to treat malaria, but WHO recommended its association with another antimalarial agent in order to avoid apparition of new resistant strains. Because the short half-life of Artemisin (approx. 2 h), its association with another antiplasmodial agent is encouraged, particularly with those having a long half-life. It is also of interest to formulate these antiplasmodial agents as extended released forms, particularly when associated with Artemisinin.

Several alkaloid producing plants, such as Guiera senegalensis (Ancolio et al., 2002), Strychnos usambarensis (Frederic et al., 1999), Balanites rotundifolia (Asrade et al., 2017), have some antimalarial (antiplasmodial) properties, but Peschiera fuchsiaefolia (Pf) was selected because this plant is low cost, non-toxic and already used by indigenous peoples in certain countries. The Pf is a tree from Apocynaceae family, also known in Brazil by the name «leiteira» because of the presence of latex in the plant, which is currently used in traditional medicine to treat malaria in Sao Paulo and Parana states. Pf extract has been reported effective against Plasmodium falciparum and other drug resistant strains (Chowdhury et al., 2017). Used under Artemisinin-based Combination Therapy (ACT), the Pf can also enhance the effect of Artemisinin bioactive agent by acting on the multidrug-resistant proteins. The antimalaria activity of Pf extracts is mainly attributed to bisindole alkaloids, particularly voacamine (FIG. 1) and probably to its commutative action with other indole alkaloids present in the extract such as voacamidine, affinisine, tabernamine.

Like most of alkaloids, the Pf extract is low water-soluble, which makes it difficult to formulate and limits the bioavailability in the bloodstream. This is why the controlled release (long-lasting) forms are appropriate to overcome these problems. A key challenge is to choose the adequate matrix for controlled delivery and protect the active agents against the stomach acidity and to transport them to the intestinal tract where the absorption sites are. Such formulation will improve the bioavailability, reduce fluctuations in drug level, enhance safety by decreasing side effects and increase patient compliance. Currently, there are no sustained release form of antimalarial alkaloid extract, such as Pf. Also, there are no rapid methods for quantification of Pf, mainly during dissolution assay.

Thus, an improvement of alkaloids solubility would be of benefit to enhance their bioavailability and to facilitate a controlled release formulation. According to an embodiment, the present invention aims to improve the solubility of the antimalarial alkaloid extract, such as Pf by complexation with carboxylated polymer having carboxyl groups, such as for example carboxymethylstarch (CMS), or carboxymethylcellulose (CMC) which will also permit to formulate the oral tablet dosage forms with extended release time and better stability of the antimalarial alkaloid extract, such as Pf. It was found that coating or complexation affect physical properties but not the biological activity of the antimalarial alkaloid extract Pf. Thus, Pf could be associated with other antimalarial drugs such as Artemisinin to elaborate controlled release formulations for an ACT.

The solubility of molecules (maximal quantity of a solute that can be dissolved in a certain quantity of solution at a specific temperature) remains one of the key factors in drug formulation, determinant to achieve desired concentration of drug in circulation for the pharmacological response. One of the techniques to enhance the water solubility and therefore the dissolution rate of poorly water-soluble drugs includes complexation.

As the present invention aims to enhance the solubility of antimalarial alkaloid extract such as Pf by complexation through ionic interaction with carboxylated polymer having carboxyl groups, in order to facilitate the sustained release formulation and to elaborate a rapid method to quantify Pf useful to follow its kinetic release profile. A comparative study of complexed-Pf with maltodextrin coated Pf is carried out to highlight the advantages of CMS.

According to an embodiment of the present invention, the carboxylated polymer may be selected from the group consisting of carboxymethylcellulose, carboxymethylstarch, or combinations thereof.

According to an embodiment of the present invention, the carboxylated polymer may have a degree of substitution of about 0.1 to about 1.0, or from about 0.1 to about 0.9, or from about 0.1 to about 0.8, or from about 0.1 to about 0.7, or from about 0.1 to about 0.6, or from about 0.1 to about 0.5, or from about 0.1 to about 0.4, or from about 0.1 to about 0.3, or from about 0.1 to about 0.2, or from about 0.2 to about 1.0, or from about 0.2 to about 0.9, or from about 0.2 to about 0.8, or from about 0.2 to about 0.7, or from about 0.2 to about 0.6, or from about 0.2 to about 0.5, or from about 0.2 to about 0.4, or from about 0.2 to about 0.3, or from about 0.3 to about 1.0, or from about 0.3 to about 0.9, or from about 0.3 to about 0.8, or from about 0.3 to about 0.7, or from about 0.3 to about 0.6, or from about 0.3 to about 0.5, or from about 0.3 to about 0.4, or from about 0.4 to about 1.0, or from about 0.4 to about 0.9, or from about 0.4 to about 0.8, or from about 0.4 to about 0.7, or from about 0.4 to about 0.6, or from about 0.4 to about 0.5, or from about 0.5 to about 1.0, or from about 0.5 to about 0.9, or from about 0.5 to about 0.8, or from about 0.5 to about 0.7, or from about 0.5 to about 0.6, or from about 0.6 to about 1.0, or from about 0.6 to about 0.9, or from about 0.6 to about 0.8, or from about 0.6 to about 0.7, or from about 0.7 to about 1.0, or from about 0.7 to about 0.9, or from about 0.7 to about 0.8, or from about 0.8 to about 1.0, or from about 0.8 to about 0.9, or from about 0.9 to about 1.0, or about ≥0.6. or about 0.1, or about 0.2, or about 0.3, or about 0.4, or about 0.5, or about 0.6, or about 0.7, or about 0.8, or about 0.9, or about 1.0. The carboxymethylcellulose may have a degree of substitution of about 0.7 to about 0.8, and preferably 0.8. The carboxymethylstarch may have a degree of substitution of about 0.1 to about 0.3, preferably 0.27.

In an embodiment, the carboxylated polymer and the antimalarial alkaloid extract are present in a ratio of from about 10:90 to about 1:99, or from about 9:91 to about 1:99, or from about 8:92 to about 1:99, or from about 7:93 to about 1:99, or from about 6:94 to about 1:99, or from about 5:95 to about 1:99, or from about 4:96 to about 1:99, or from about 3:97 to about 1:99, or from about 2:98 to about 1:99, or from about 10:90 to about 2:98, or from about 9:91 to about 2:98, or from about 8:92 to about 2:98, or from about 7:93 to about 2:98, or from about 6:94 to about 2:98, or from about 5:95 to about 2:98, or from about 4:96 to about 2:98, or from about 3:97 to about 2:98, or from about 10:90 to about 3:97, or from about 9:91 to about 3:97, or from about 8:92 to about 3:97, or from about 7:93 to about 3:97, or from about 6:94 to about 3:97, or from about 5:95 to about 3:97, or from about 4:96 to about 3:97, or from about 10:90 to about 4:96, or from about 9:91 to about 4:96, or from about 8:92 to about 4:96, or from about 7:93 to about 4:96, or from about 6:94 to about 4:96, or from about 5:95 to about 4:96, or from about 10:90 to about 5:95, or from about 9:91 to about 5:95, or from about 8:92 to about 5:95, or from about 7:93 to about 5:95, or from about 6:94 to about 5:95, or from about 10:90 to about 6:94, or from about 9:91 to about 6:94, or from about 8:92 to about 6:94, or from about 7:93 to about 6:94, or from about 10:90 to about 7:93, or from about 9:91 to about 7:93, or from about 8:92 to about 7:93, or from about 10:90 to about 8:92, or from about 9:91 to about 8:92, or from about 10:90 to about 9:91, or about 10:90, or about 9:91; or about 8:92, or about 7:93, or about 6:94, or about 5:95, or about 4:96, or about 3:97, or about 2:98, or about 1:99, and preferably about 4:96. The carboxylated polymer:antimalarial alkaloid ratio is dependent on the viscosity of the complex solution. In embodiments with a ratio 10:90, the complex is manipulable. In other embodiments, the ratio will also be possible provided that the molecular weight of the carboxylated polymer is sufficiently low to provide a suitable viscosity to prepare powders (e.g. 200 cP).

A previous formulation approach consisted in coating Pf with a maltodextrin (MD) which is a commercial excipient (oligosaccharide of 3 to 17 D-glucose units chain lengths).

Carboxymethylstarch (CMS) and carboxymethylcellulose (CMC) excipients are able to ensure high loading of bioactive agents and to offer gastric resistance, adequate delivery and a long-term stability. In addition, CMS and CMC are classified as GRAS (Generally Recognized as Safe), inexpensive and easy to obtain.

According to embodiments, the controlled release complex of the present invention may be prepared with various antimalarial alkaloid extract, which may be selected from the group consisting of alkaloid extracts from Guiera senegalensis, Strychnos usambarensis, Balanites rotundifolia and Peschiera fuchsiaefolia. Preferably, it is selected from Peschiera fuchsiaefolia (Pf).

According to another embodiment, there is disclosed a solid oral dosage form comprising the controlled release complex of the present invention and pharmaceutically acceptable excipients. In embodiments, the solid oral dosage form of the present invention may comprise known pharmaceutically acceptable excipients, such as binding agents, (e.g. sucrose, lactose, starches, cellulose, microcrystalline cellulose, xylitol, sorbitol, mannitol, gelatin, hydroxypropyl cellulose (HPC), hydroxypropyl methylcellulose—HPMC, polyvinylpyrrolidone (PVP), polyethylene glycol (PEG)), and lubricants (e.g. talc, silica, magnesium stearate, vegetable stearin, or stearic acid). In embodiments, the oral dosage form is monolithic—i.e. formed as a single dosage made from compressed powder material.

According to another embodiment, there is disclosed a solid oral dosage form for the controlled release of an antimalarial drug combination, comprising the controlled release complex of the present invention, an antimalarial drug, and pharmaceutically acceptable excipients.

In embodiments, the antimalarial drug may be selected from the group consisting of quinine and artemisinin, artesunate, artemether, arteether, dihydroartemisinin, and artelinate. Preferably, the antimalarial drug is artemisinin. In embodiments, the solid oral dosage form for the controlled release of an antimalarial drug combination of the present invention may comprise known pharmaceutically acceptable excipients, such as binding agents, (e.g. sucrose, lactose, starches, cellulose, microcrystalline cellulose, xylitol, sorbitol, mannitol, gelatin, hydroxypropyl cellulose (HPC), hydroxypropyl methylcellulose—HPMC, polyvinylpyrrolidone (PVP), polyethylene glycol (PEG)), and lubricants (e.g. talc, silica, magnesium stearate, vegetable stearin, or stearic acid). In embodiments, the oral dosage form is monolithic—i.e. formed as a single dosage made from compressed powder material. According to another embodiment, the dosage form is multilayered, preferably bilayered.

According to another embodiment, the oral dosage form may further comprise an additional carboxylated polymer, uncomplexed with the antimalarial alkaloid extract.

### Use of the Formulations of the Present Invention

In another embodiment there is disclosed a method of treating malaria comprising administering to a subject in need thereof an effective amount of the solid dosage form of the present invention.

In another embodiment there is disclosed a use of the solid dosage form of the present invention for the treatment of malaria in a subject in need thereof.

In another embodiment there is disclosed a use of the controlled release complex of the present invention, for the manufacture of a medicament for treatment of malaria in a subject.

In another embodiment there is disclosed a use of the solid oral dosage form of the present invention, for the manufacture of a medicament for treatment of malaria in a subject.

According to yet another embodiment, there is disclosed a process for the preparation of a controlled release complex of an antimalarial alkaloid extract comprising the steps of contacting a carboxylated polymer having carboxyl groups and having a degree of substitution of about 0.1 to about 1.0 suspended in water with the antimalarial alkaloid extract dissolved in a solvent-water medium.

In some embodiments, the solvent-water medium is a hydroalcoholic medium, for example 60% v/v ethanol in water.

In some embodiments, the carboxylated polymer is selected from the group consisting of carboxymethylcellulose (CMC), carboxymethylstarch (CMS), or combinations thereof.

The carboxymethylcellulose may have a degree of substitution of about 0.7 to about 0.8. preferably about 0.8. The carboxymethylstarch may have a degree of substitution of about 0.1 to about 0.3, preferably a degree of substitution of about 0.27.

The carboxylated polymer and the antimalarial alkaloid extract may be present in a ratio of about 4:96.

The antimalarial alkaloid extract may be selected from the group consisting of an alkaloid extract from Guiera senegalensis, Strychnos usambarensis, Balanites rotundifolia and Peschiera fuchsiaefolia and preferably Peschiera fuchsiaefolia.

The present invention will be more readily understood by referring to the following examples which are given to illustrate the invention rather than to limit its scope.

### Example 1

### Complexation of PF with CMS

### 1.1 Materials

Peschiera fuchsiaefolia (Pf) extract was from Yerbalatina phytoactives(Colombo, PR, Brazil) and Artemisinin (Arte) from Changsha Inner Natural Inc., (Changsha, Hunan, China). Maltodextrin was purchased from Aldrich chemical Company Inc (Milwaukee, WI, USA), High amylose corn starch (Hylon VII) was from Ingredion Incorporated (Bridgewater, NJ, USA). The ABTS [2,2-azino-bis (3-ethylbenzothiazoline-6-sulfonic acid)] diammonium salt (purity 98%), and Trolox (6-hydroxy-2,5,7,8-tetramethyl-chroman-2-carboxylic acid) 97%, were from Sigma-Aldrich (St. Louis, MO, USA). Hydroxypropylmethylcellulose (HPMC) was from Dow Chemical Company (Midland, MI, USA) and Sodium chloride (99%) was from Anachemia (Montreal, QC, Canada). All other reagents were of analytical grade and used without further purification.

### 1.2 Methods

### 1.2.1 Synthesis of CMS

Carboxymethylstarch (CMS) at different degrees of substitution (DS) was prepared from high amylose starch first gelatinized at 65° C. in NaOH medium followed by treatment with sodium monochloroacetate (SMCA) as described by Assaad and Mateescu (2010). Practically, three CMS (CMS1, CMS2 and CMS3) with different DS were obtained varying the ratio starch/SMCA.

The DS was determined by titrimetric methods as described by Le Tien et al. (2004) with some modifications, as follows: the carboxyl groups of the CMS (1.0 g) were first converted into the acidic (protonated) form by dispersion of the modified polymer (CMS) in ethanol (70%) containing 1.0 M HCl. The protonated CMS was filtered, washed several times with ethanol/distilled water (80:20) in order to completely remove the acid in excess, and then precipitated and dried with pure acetone. Finally, an aliquot of 100 mg of protonated CMS powders was suspended in 100 mL distilled water and titrated with a 0.05 M sodium hydroxide. The amount of carboxyl groups (DS) was calculated as described by Stojanovic et al. (2005).

1.2.2 Pf Coating with Maltodextrin

The Peschiera fuchsiaefolia (Pf) pure extract was coated with maltodextrin (MD) to obtain the MD/Pf. Practically, an amount of 50 mL of maltodextrin (20% in water) was slowly added in 450 mL of Pf (20% dissolved in alcohol 60%). This solution with a final ratio Pf/maltodextrin (w/w) of 90/10 was maintained under agitation at room temperature until spray-drying (Büchi Mini Spray Dryer B-290, Büchi Labortechnik AG, Flawil, Switzerland) using following parameters: i) inlet temperature 150° C.; ii) outlet temperature 100° C.; iii) spray flow 500 L/h, and iv) aspirator setting at 38 m³/h. During the drying process, the MD excipient/Pf suspension was continuously gently stirred to prevent sedimentation and to obtain a homogeneous powder.

### 1.2.3 Preparation of CMS-Pf Complexes

Ionic interaction between CMS and Pf resulting in Pf-CMS complexation was first evaluated in relation to the DS. At an aqueous filtered (0.45 μm) solution of Pf (3 g in 200 mL), an equivalent quantity of CMS (3 g) was added. After homogenization and incubation under stirring at room temperature (30 min), CMS (free form or complexed with Pf) was precipitated by adding methanol in excess; then, decantation product was collected and washed with methanol on filter paper. The obtained product was dried overnight at room temperature and then at 70° C. for 24h. Similar operation was carried out with the three different CMS (CMS1, CMS2 and CMS3). The quantification of Pf complexed with CMS in different Pf-CMS complexes (Pf-CMS1, Pf-CMS2 and Pf-CMS3) was carried out by UV (λ 280 nm). Its antioxidant capacity was also measured via the discoloration of blue-green ABTS^{•+} radical cations (Konan et al., 2016). The CMS that fixed the largest amount of Pf (% w/w) was selected for further investigation.

CMS was dispersed at 8.5% in water. After homogenization and obtaining a homogenous suspension, an amount of 50 mL of CMS suspension was slowly added in 450 mL of Pf 21.5% previously dissolved in ethanol (60%). After homogenization of the solution, the complexed CMS-Pf powders were obtained by spray-drying using the same parameters as previously (MD/Pf coating). The final ratio Pf/CMS was about of 96/4.

### 1.2.4 Formulation of CMS-Pf Complexes as Monolithic Tablets

The formulations consisted in powders of 450 mg of CMS-Pf complex powder mixed with 120 mg of CMS (CMS1, CMS2 or CMS3) and 100, 120, 150 or 200 mg of hydroxypropyl methylcellulose (HPMC) as a binding agent. An amount of 15 mg of magnesium stearate was used as lubricant. Tablets were obtained by direct compression of mixed powders at 2.3 T/cm².

### 1.2.5 Characterization of Pf Complexes

### 1.2.5.1 Fourier Transform Infrared (FTIR) Spectroscopy

The FT-IR spectra of samples (Pf, MD, CMS, MD/Pf and CMS-Pf under powder or tablet form) were performed on Thermo Scientific Nicolet 6700/Smart iTR (Madison, WI, USA) equipped with a deuterated triglycinesulfate-KBr) detector and a diamond smart attenuated total reflection platform. Before the spectral analysis, the crystal was cleaned with ethanol and a background spectrum was acquired. The parameters were: number of scans, 32; resolution, 4 cm⁻¹. All the analyses were carried out at room temperature.

### 1.2.5.2 X-Ray Diffraction (X-RD)

The diffraction patterns of different samples (Pf, MD, CMS, MD/Pf and CMS-Pf) were recorded using a Siemens D-5000 diffractometer (Munich, Germany) SOL-X detector with a Cobalt cathode in reflectance mode at a wavelength of 1.789 Å. The diffractograms, registered over an angular range of 20 from 0° to 30° and a scan rate of 2°/min, were treated using the Diffracplus software.

### 1.2.5.3 Scanning Electron Microscopy (SEM)

The microstructures of samples (Pf, MD, MD/Pf and CMS-Pf) were examined with a JSM-6010LV InTouchScope™ (SEM) (JEOL, Tokyo, Japan) using a secondary electron image detector. Small amount of powder was mounted on a metal stub with an adhesive and covered under vacuum with a fine layer of gold in a sputter coater BIO-RAD E5200 (Bio-Rad Laboratories Ltd., London, UK). The microphotographs were obtained by accelerating voltages of 1.5 kV and high vacuum.

### 1.2.6 Stability of Pf, MD/Pf and CMS-Pf in Simulated Gastric and Intestinal Fluids

Structural stability of different samples was evaluated after incubation in simulated gastric fluid (SGF) and followed in simulated intestinal fluid (SIF).

Monolithic tablets of Pf, MD/Pf or CMS-Pf (without other ingredients) were incubated (0-2 h at 37° C.) in SGF and/or in SIF (0-2 h at 37° C.). The samples were then dried and the structure was analyzed by FTIR and the antioxidant activity was measured by the ABTS method (as described below).

1.2.7 Antioxidant Proprieties of Peschiera fuchsiaefolia (Alkaloid) Extracts

Antioxidant activity of Peschiera fuchsiaefolia extracts was evaluated before and after coating with MD and complexation with CMS. In fact, after dispersion of samples (Pf, MD/Pf and CMS-Pf) in different media (distilled water, SGF and SIF), the antioxidant capacity of samples was evaluated by spectrophotometric method, as previously described (Konan et al., 2016). Similarly to the TEAC classical assay, this ABTS test is based on the scavenging of the blue-green ABTS^{•+} radical cations which was generated by electrolysis of the ABTS solution (Konan et al., 2016). Practically, a volume of 6.5 mL of 140 μM ABTS (in 0.85% NaCl) was electrolyzed (400 Volts, 10 mA) during 20 sec to generate the ABTS^{•+} radical. The electrodes of platinum were 2.12 mm diameter, 25.33 mm length with inter-electrode space of 26.36 mm (measured directly with an Electronic Digital Caliper (Docap Corp., St-Laurent, Quebec, Canada) for the range 0-200 mm with readings at a precision of tens microns).

For scavenging capacities of samples analysis, the electrolysis induced ABTS^{•+} solution (950 μL) was mixed with 50 μL of 0-200 μM Trolox for calibration or with 50 μL of the tested samples (Pf, MD/Pf or Pf-CMS) and their absorbances were recorded at 734 nm, after 1 min of incubation. The decrease of absorbency due to the antioxidant capacity reflects the scavenging of the free radical (ABTS^{•+}). The abilities of Pf, MD/Pf and Pf-CMS to scavenge the ABTS^{•+} radical cations were compared to that of Trolox (hydrosoluble structural analogue of vitamin E) used as reference antioxidant. The results are expressed in mM of Trolox equivalence per mg of product. Each measurement consisted of at least five readings and the values represented the mean±standard deviations.

### 1.2.8 Solubility Assay of Powders of pf and Pf Complexes

Solubility of Pf (MD/Pf, CMS-Pf and Pf) in distilled water, in SGF and in SIF was evaluated and defined as described in Pharmacopeia and National Formulary (USP-NF) (USP, 2017a). Each sample (Pf, MD/Pf or CMS-Pf) was added carefully to 100 mL of medium (water, SGF or SIF under stirring at 37° C.) until saturation and accumulation of solid matter. Thereby, the quantity of Pf necessary to saturate this volume was obtained and the approximate solubility range (mg/mL) was determined. Then, the solubility was quantified according Cano-Chauca et al. (2005) and by a new approach, based on a specific capacity of the sample of interest.

### 1.2.8.1 Approach According to Cano-Chauca et al. (2005)

This study was performed by adding excess of Pf, MD/Pf or CMS-Pf (20 g) in 100 ml of different media. The saturated solution prepared with each medium (distilled water, SGF or SIF) was shaken at 37° C. for 24 h. The mixture was centrifuged at 2000 g for 10 min; the supernatant was filtered using 0.45 μm Millipore filter. An aliquot of 30 mL was pre-weighed in Petri dishes, oven-dried at 105° C. for 5 h, cooled down in a desiccator and weighed. The solubility (mg/mL) was calculated as the weight difference for determining the amount of Pf (mg) per volume (mL) of solution.

1.2.8.2 Solubility (mg/mL) Determined by Spectrophotometric Dosage (Absorption at 280 nm) and by Antioxidant Capacity of Samples

Saturation was obtained with sample (2 g) in medium (10 mL). Incubation takes place for 5 hours and solution medium was centrifuged. After filtration of supernatant, the concentration of Pf was determined by UV at 280 nm in an aliquot (1 mL), and on the other hand, the antioxidant activity of an aliquot (10 μL) was evaluated. Concentrations (mg/mL) were determined using standard curves as functions of sample concentrations (mg/mL) previously established. Standard curves were established with different concentrations of Pf and derivatives (MD/Pf and CMS-Pf) from 0 to 1 mg/mL in different media.

### 1.2.9 Dissolution Assay

Dissolution assays of formulated monolithic tablets were done in simulated gastric fluid (SGF, pH 1.5) for 2 h and continued in simulated intestinal fluid (SIF, pH 6.8) as referred by USP method 40 (USP, 2017a).

The Pf (Pf, MD/Pf or CMS-Pf) release kinetics from different formulations (Pf, MD/Pf and CMS-Pf monolithic tablet dosage form) were recorded at 100 rpm and 37° C. using a Distek dissolution system 2100A (Betatek Inc., Markham, ON, Canada). At predetermined intervals, an established volume of samples (1 mL) was withdrawn from dissolution media, filtered (0.45 μm) and the released Pf was quantified by UV measurement at 280 nm.

### 1.3. Results and Discussion

### 1.3.1 Results

### 1.3.1.1 The Degree of Substitution (DS) of Synthesized CMS

DS (% carboxymethyl group/glucose unit) depended of the three CMS synthesized (CMS1, CMS2 and CMS3), and were respectively 17.69±0.19 (0.1769±0.0019), 22.01±0.17 (0.2201±0.0017) and 27.03±0.15% (0.2703±0.0015) for CMS1, CMS2 and CMS3.

### 1.3.1.2 Characterization of MD/Pf and CMS-Pf

### 1.3.1.2.1 CMS-Pf Complexation Yield

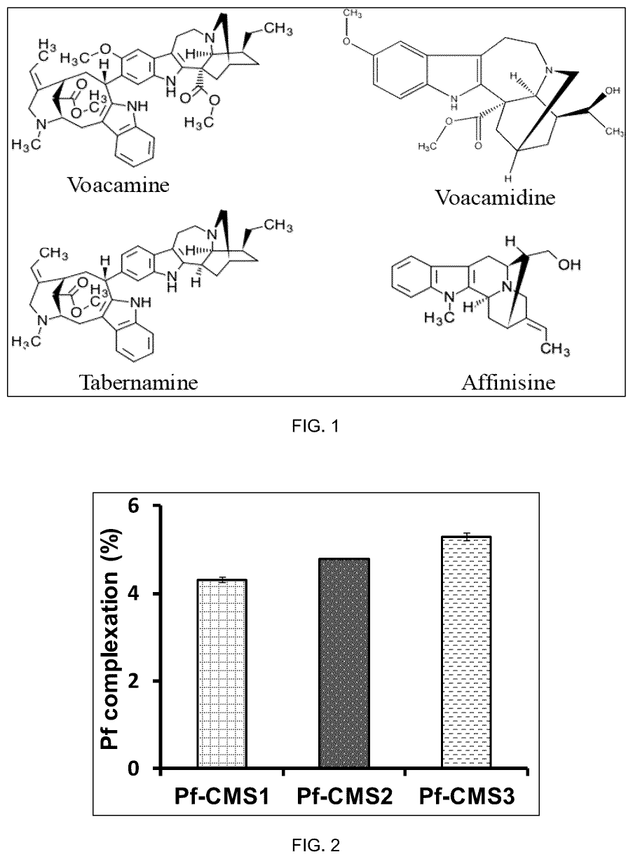

CMS-Pf complexation yield increases with DS of the CMS (FIG. 2).

### 1.3.1.2.3 FTIR Analysis

FTIR spectra showed significant differences between CMS (CMS1, CMS2 and CMS3) and native starch (high amylose starch).

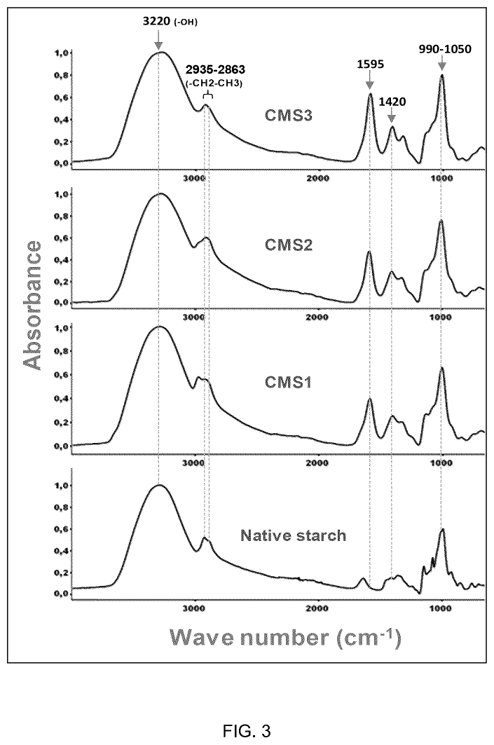

In native starch spectrum (FIG. 3), an absorption band at 1640 cm⁻¹ is ascribed to hydroxyl (—OH) groups. After carboxymethylation as CMS, the appearance of new absorption bands at 1595 and 1415 cm⁻¹ are assigned to asymmetric and symmetric stretching vibrations of carboxylate anion groups. The absorption intensity at 1595 cm⁻¹ is greater for CMS3 which is probably due to its higher DS.

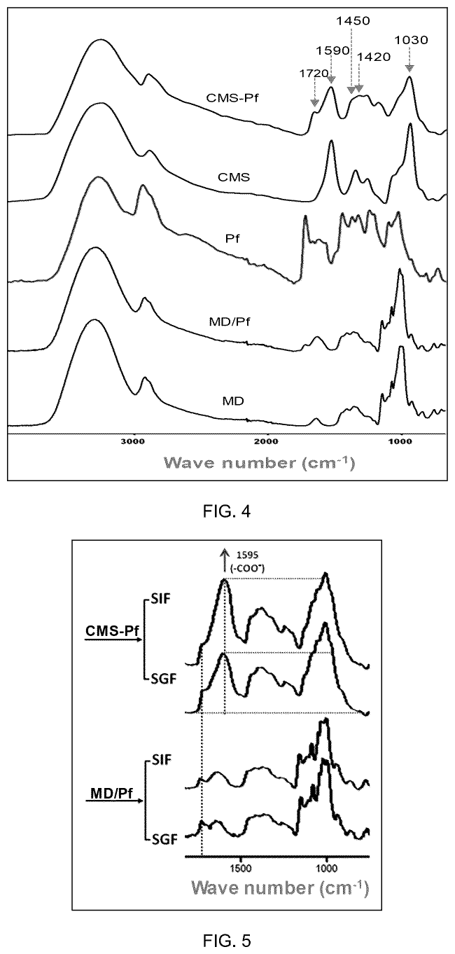

FIG. 4 also shows FTIR spectra of MD and of Pf native and coated with maltodextrin (MD/Pf), or complexed with CMS as CMS-Pf.

For MD, the IR spectral profile presents the pattern similar to that of native starch, with O—H absorption bands at 3300, at 1640 cm⁻¹ and at 1010 cm⁻¹.

Native Pf exhibits absorption intensities for C—H stretching groups in the region 2935-2863 cm⁻¹. Absorption bands at 1750, 1700, 1610 cm⁻¹ are potentially attributed to C═O (from esters and acids groups) and C═C of Pf. The spectral region 1600-1300 cm⁻¹ is mainly assigned to aromatic C═C (symmetric stretching), N—H and C—N vibrations.

When Pf is coated with MD, a major difference was observed by a reduction of absorption of intensities of bands in spectral region 1750-1550 cm⁻¹, probably due to the coating of MD which surrounds and hides the Pf. However, two absorption bands at 1750 and 1680 cm⁻¹ were still noticed and possibly attributed to C═O (from amides), C═C and to C═N stretching vibrations of Pf.

For Pf complexed with CMS, a decrease of the absorption band at 1595 cm⁻¹ was observed when compared to CMS alone, probably due to interactions between carboxylate groups of CMS and amine groups from Pf with an absorption band appearing at 1470 cm⁻¹. A new band appeared at 1720 cm⁻¹ which is assigned to C═O (acid and carbonyl) and probably to C═N (from oxidation of nitrogen functions) stretching vibration of Pf.

After incubation in SGF (2h) and then in SIF (2h), no marked differences (FIG. 5) in the FTIR patterns were found for the MD/Pf. For CMS-Pf complex, when transferred from SGF in SIF, an increase in intensity of carboxylate bands (1595 cm⁻¹) was observed (FIG. 5). When CMS was transferred in intestinal medium (pH 6.8), the carboxylic groups previously protonated during the 2 h transit in SGF (pH 1.5) are deprotonated with a further increase of this absorption band at 1595 cm⁻¹ ascribed to carboxylate ions.

### 1.3.1.2.4 X-Ray Diffraction (X-RD) of Pf Forms

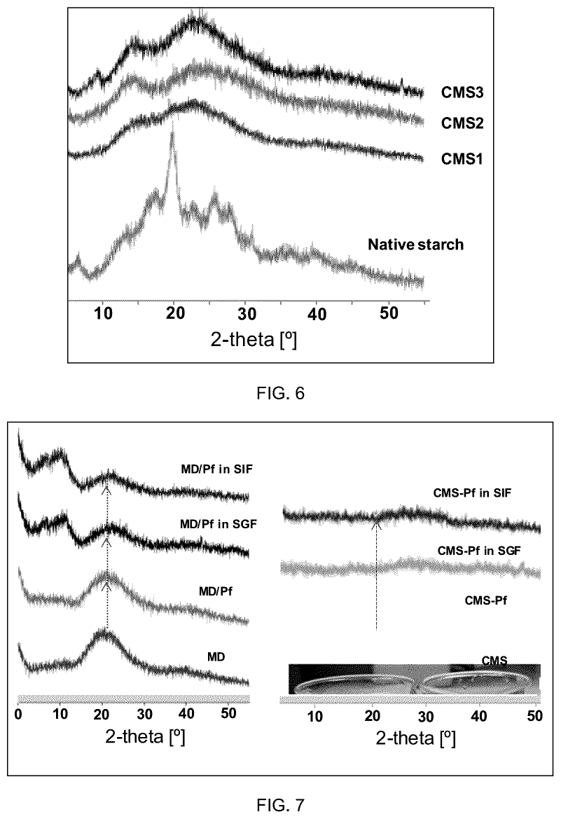

X-RD analysis showed for native starch an organized structure with a higher-order degree and several crystalline domains whereas by carboxymethylation, starch loses its crystallinity (FIG. 6). Indeed, native starch diffractogram presents a predominant double helix B-structure characterized by diffraction bands with 2-theta angles at 14°, 17°, 20°, 26° and 28° and a minor single helix V-structure with bands at 14° and 22°. After carboxymethylation, starch diffractogram presented only 2 main bands with 2-theta angles at 14° and 22° attributed to single helix V-structure. However, CMS bands were larger and diffuse with lesser crystallinity than that of native starch. When compared CMS at different DS, it appears that CMS with higher DS presents a V-structure more organized than the others at lower DS.

For maltodextrin (MD alone) and Pf coated with maltodextrin (MD/Pf), their diffractogram presented a similar profile with a main 2 theta angle at 21°. After incubation of the MD/Pf aggregate in SGF and in SIF, there is an appearance of a new 2 theta angle at 14° whereas the angle at 21° remained generally unchanged (FIG. 7).

When complexed with Pf, the CMS structures lose most of crystallinity with a decrease in intensity of bands. The bands became broader and tended to disappear. Similar diffractogram profiles were observed after incubation of CMS-Pf in SGF and in SIF, with large peaks at lower intensities.

### 1.3.1.2.5 Scanning Electron Microscopy of the Pf Complexes

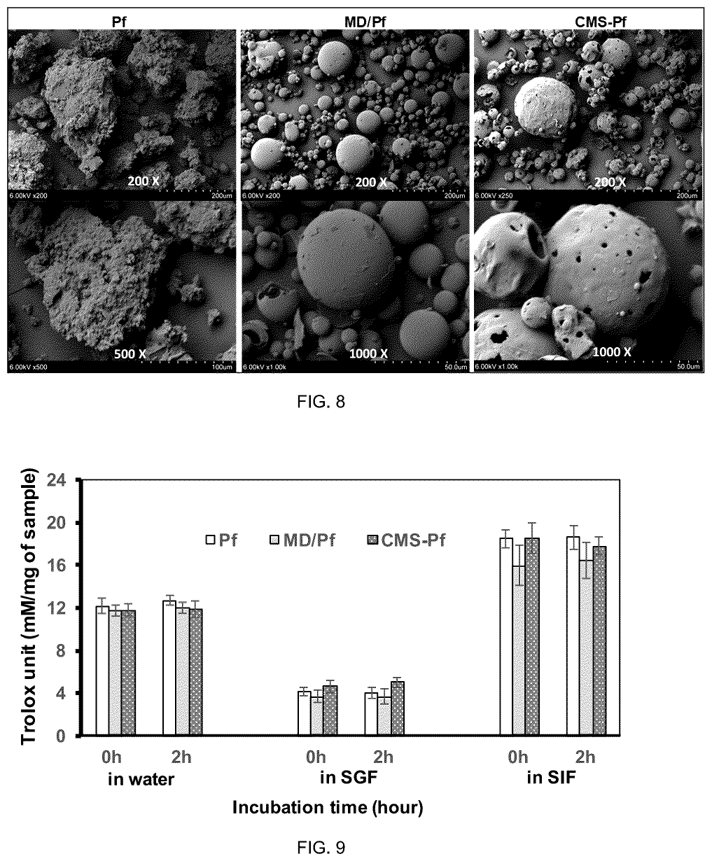

SEM of Pf coated with MD or complexed with CMS showed granules with spherical shape and variable sizes (FIG. 8). In fact, CMS-Pf granules presented spherical surfaces with numerous holes whereas the surface of MD/Pf was smoother with only a few holes.

1.3.1.3 Antioxidant Activity of Peschiera fuchsiaefolia (Alkaloid) Extracts

The antioxidant capacities of free Pf and of coated Pf with MD (MD/Pf) and complexed as CMS-Pf after and before incubation in SGF and in SIF were evaluated by the fast Trolox Equivalent Antioxidant Capacity (TEAC) assay. Results were expressed in Trolox equivalent units (mM) per mg of sample, not in μM/g which is generally used. Conversion in μM/g would be by multiplying different values by 10⁶.

Antioxidant activities were significantly higher in SIF than in nanopure water or in SGF (with activities markedly decreased). The three samples presented similar antioxidant activity before and after 2 h incubation in the respective mentioned media.

After 2 h incubation in SGF, CMS-Pf presented a slightly higher antioxidant activity significantly different when compared with those of Pf and MD/Pf. This behavior is probably due to the protonation of carboxylic groups, which are thus compacted and protected in gastric acidity. No changes in antioxidant properties of Pf were induced by MD or CMS excipients (FIG. 9).

### 1.3.1.4 Solubility of Complexes

After filtration of saturated solutions of Pf, of coated Pf (MD/Pf) or of CMS-Pf in different media (water, SGF and SIF), they presented different color characteristics and intensities of alkaloids. They were darker in SGF and SIF than in nanopure water and more pronounced for MD/Pf and complexed CMS-Pf than for free Pf saturated solution with the highest intensities for CMS-Pf and when it was dissolved in SGF medium (FIG. 13).

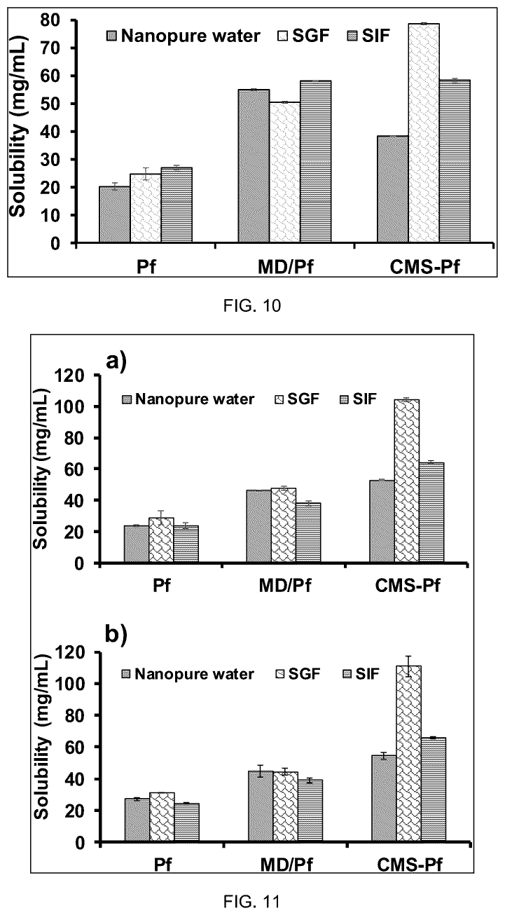

Solubility values (mg/mL) in different media (nanopure water, SGF and SIF) of the coated Pf and of the CMS-Pf complex are markedly higher than free Pf extract, regardless determination approach of the solubilization (FIGS. 10 and 11).

Contrary to the intensity of their colorations, the solubility as evaluated by the drying approach was higher for the coated MD/Pf than that of CMS-Pf complex in water and in SGF media (FIG. 10). In SIF, the solubility of CMS-Pf was higher due to strong ionization of CM groups.

Solubility values obtained using the new approach correlate with coloration intensity of different maxima concentration solutions (FIGS. 11 and 13).

Solubilities of each sample were significantly different in the media (water, SGF, or SIF), except for the coated MD/Pf complex (FIG. 11a). These solubility values are higher with the coated Pf or with the CMS-Pf complex, with the CMS-Pf complex solubility in SGF being the highest (FIG. 11b). In the three media, solubility values were similar for the two-different duration (5 h and 24 h) of incubation (FIG. 11). In reference to the first approach, saturated solution was obtained after incubation 24 h, but another saturated solution was prepared during only 5 h incubation. Regardless of the incubation duration (5 h or 24 ), no significant difference was detected (FIG. 11). Overall, this approach permitted to save about 24 h and may be considered a fast method.

### 1.3.1.6 Dissolution Assay

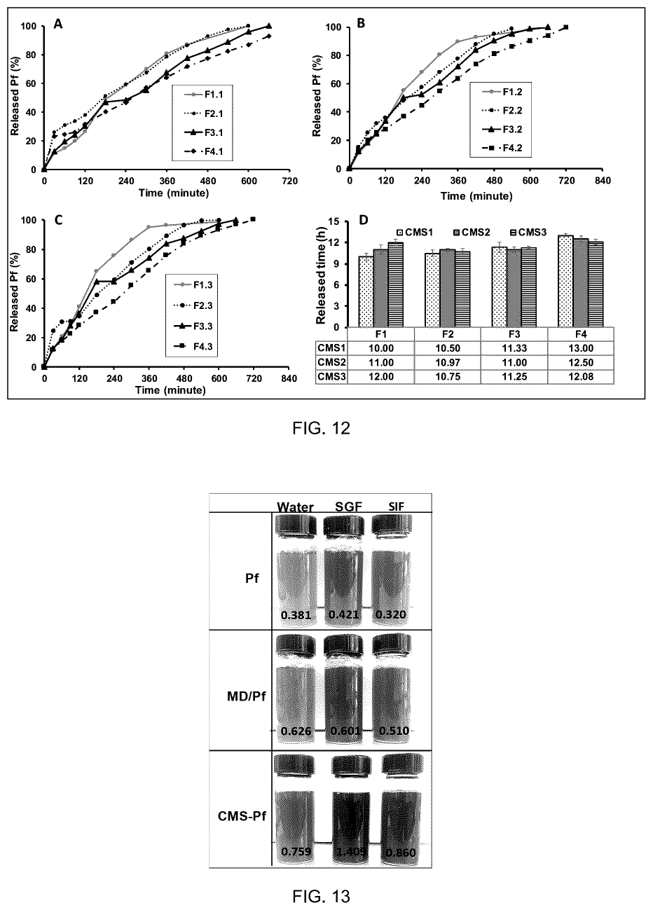

The release kinetics of CMS-Pf from different formulations (FIG. 12) showed that Pf was slowly and gradually released in SGF and in SIF (FIG. 12A-C). After 2 h in SGF, about 100-150 mg were released and the complete release of Pf (450 mg) was achieved after 10-13 h (FIG. 13D). Total released time did not vary significantly with DS of CMS used as matrix.

### 1.3.2 Discussion

The samples of interest were MD/Pf and CMS-Pf. The MD/Pf materials obtained by coating without other treatment were unstable in time and rapidly released the Pf. To overcome these limitations, the cationic Pf alkaloid was complexed with the anionic CMS excipient.

Pf is mainly composed by bis-indole alkaloids containing amine groups which are under secondary (from Voacamin, Voacamidin, Tabernamine), tertiary (from Voacamin, Voacamidin, Tabernamine, Affinisine) and possible quaternary forms. The carboxylic negative charges of CMS can ionically interact with amine groups of Pf to form a CMS-Pf complex via ionic interaction between carboxylate groups of CMS and amine groups of Pf.

The new absorption bands of CMS at 1595 and 1415 cm⁻¹ (FIG. 3) are ascribed to asymmetric and symmetric stretching vibrations of carboxylate anions (—COO⁻) and the new band at 1725 cm⁻¹ was assigned to carboxyl groups under protonated form.

For MD (FIG. 4), stretching vibration at 3300 and 1640 cm⁻¹ corresponded to O—H groups, and at 1010 cm⁻¹ for C—O groups. Assembled in MD/Pf coating complex, the new band found at 1725 cm⁻¹ was ascribed to carbonyl groups of Pf. The MD/Pf aggregate seems to maintain its conformation in SGF and in SIF as supported by the lack of differences after incubation in these media (FIG. 5).

Concerning CMS-Pf complex, a band at 1590 cm⁻¹ assigned to carboxylate groups was decreased upon complexation suggesting interactions between carboxylate groups of CMS and amine groups of Pf. Compared to CMS alone, the slightly decreased intensity of the absorption at 1595 cm⁻¹ would suggest that after complexation with Pf, an amount of free carboxylate still remained available. In SGF, the protonation of carboxylate to carboxylic acid groups and a possible interaction with the ionized ammonium groups of Pf may explain the reduced intensity of carboxylate band at 1595 cm⁻¹. Transferred in the SIF (FIG. 5), the increase in intensity of carboxylate bands (1595 cm⁻¹) seems due to the deprotonation of carboxylic acid group.

The X-ray patterns indicated for the coated MD/Pf a structure similar to that of MD, but with a lesser order after incubation in SGF and SIF solutions (FIG. 7) suggesting that CMS via its carboxylic groups when interacting with amine groups will lose hydrogen interactions and crystallinity in favor of an amorphous structure and an improved solubility in SGF and SIF media.

The MD used to coat Pf is able to partially or completely cover Pf granules explaining the smooth surface observed in SEM for MD/Pf (FIG. 8).

The in vitro determination of antioxidant activity of Pf and of its coated and complexed forms revealed that Pf is a very good antioxidant agent (FIG. 9). The Pf, as many other alkaloids, acts efficiency at physiological pH 6.8.

For an equal mass, antioxidant activity of Pf was moderately low but in the same order of magnitude, when compared to the Trolox (Table 1).

TABLE 1
Antioxidant activity of Pf, MD/Pf and CMS-Pf versus
the antioxidant activity of equal mass of Trolox
Antioxidant activity (% of Trolox activity)

| Sample form | Water | SGF | SIF |
|---|---|---|---|
| Pf | 15.23 ± 0.48 | 5.21 ± 0.11 | 23.11 ± 2.98 |
| MD/Pf | 14.67 ± 0.23 | 4.58 ± 0.03 | 19.95 ± 5.19 |
| CMS-Pf | 14.74 ± 0.05 | 5.78 ± 0.34 | 23.20 ± 6.27 |

The stability test carried out under tablet form at 40° C. and relative humidity (RH) 40% showed that the MD/Pf tablets were hydrated and melted. The powder in these conditions changed color from light brown to dark brown after one week. This phenomenon was possibly due to hygroscopic properties of maltodextrin which holds water in the tablet and thus favors Maillard reaction between reducing hemiacetal of maltodextrin and amine groups from Pf alkaloids. To prevent this, it is necessary to enhance the Pf stability before the formulation. In order to reduce the undesirable effects due to the Maillard reaction (the browning phenomenon), it was of interest to minimize the reactivity of alkaloid amine groups by complexation with carboxyl anion groups of the carboxymethyl starch. These interactions may also prevent undesirable interactions with other bioactive ingredients, particularly in formulation of Pf combined with other bioactive agents (e.g. artemisinin).

Since the samples are derivatives of the same Pf product, with approximatively the same color, their solubility can be compared spectrometrically from their color intensity. After filtration, photos were made with the saturated solutions of different samples (FIG. 13) and their absorbencies at 280 nm (λmax) were measured. Intensities of coloration showed higher solubility of samples in SGF and in SIF than in nanopure water. The highest solubilities in SGF were found with Pf and with CMS-Pf. Solubility of free Pf was slightly higher in SGF. These results are in agreement with x-ray pattern (FIG. 7) and morphology of samples (FIG. 8). More organized structure was correlated with a higher stability and lower solubility of the sample (FIG. 7). For instance, CMS-Pf with an amorphous structure would be more soluble than MD/Pf.

After obtaining saturated and filtered solution, the Cano-Chauca et al (2005) approach was used to determinate the weight (mass) of remaining solute in a known volume after drying process for Pf or coated MD/Pf or complexed CMS-Pf. Thus, the weight measured were not only for Pf but also from the additive coating (MD) or complexation (CMS) agents. In fact, MD is an oligosaccharide consisting of D-glucose units with chain lengths from 3 to 17 glucose units.

The MD is hydrophilic and with a much higher solubility than the alkaloid. In the MD/Pf coating, there is no binding to retain MD when incubated in an aqueous medium. In this context, during saturation, a large amount of MD would be easily dissolved and thus largely contributing to the solubility of Pf as MD/Pf. Differently, the CMS involved in CMS-Pf complexation is less soluble than MD. However, the residual mass values obtained for MD/Pf and CMS-Pf were not limited to Pf from these samples. Furthermore, CMS-Pf complex material may contain free CMS, not complexed to Pf (as showed at FIG. 3, supported by the band at 1590 cm⁻¹). It would be easily and rapidly dissolved and enhancing thus the weight of solute.

This approach does not seem appropriate for complexes, mixtures or association of nonhomogeneous samples, with various compounds with different solubilities. For this reason, a new approach is proposed to quantify the solubility of only one of compound in the complex based on a specific property of the analyzed agent. This approach may evaluate effective solubility (mg/mL) of an agent in a complex sample. With this approach, after filtration and obtaining saturated solution, the effective quantity or the effective concentration of the compound of interest (Pf) is determined based on an intrinsic capacity (or activity) of the concerned agent. In the case of Pf, of the coated MD/Pf and of CMS-Pf complexes, the absorption of indole nuclei (at λ 280 nm) and the antioxidant activity were used to evaluate Pf concentration (mg/mL) in saturated solution. This value corresponded to solubility (mg/mL).

Differing to precedent approach, results from the new proposed approach (FIG. 11) were in accordance with results indicated by coloration intensity and in conformity with samples composition (FIGS. 7 and 8) and with their behavior in SGF and SIF during incubation (FIG. 5). Since, the absorbance at 280 nm and the antioxidant activity were features of Pf only in different samples, the obtained values effectively reflect the Pf, MD/Pf and CMS-Pf solubility. With this new approach, the drying time (5 h) was avoided since the intrinsic scavenging capacity (antioxidant capacity) was directly determined after obtaining the filtered saturated solutions.

This approach consists to determine only the effective concentration (mg/mL) and thus the solubility of compound of interest in various saturated solution containing different components based on one of its distinctive activity or property. This procedure is advantageous for multicomponent samples (mixing of different compound, i.e: MD/Pf and CMS-Pf), when it is difficult to determine their solubility by measuring the dry weight per volume (as above).

Also, salts present in the dissolution media (SGF, SIF) may influence results. Therefore, it would be necessary to measure effective quantity or concentration or mass of compound of interest. In table 2 below, the solubility values obtained for each sample and were descripted in the corresponding solubility terms of the USP-NF (USP, 2017a). The coating (MD/Pf) and complexation (CMS-Pf) of Pf clearly enhanced the solubility in different media (Table 2), and consequently, the bioavailability. The Pf, sparingly soluble, became soluble in water, SGF and SIF when coated with MD. It was also soluble in water and SIF under CMS-Pf complex form, but in SGF it was freely soluble. MD/Pf and CMS-Pf were each soluble in water and in SIF, but the CMS-Pf solubility values were significantly higher than that of MD/Pf. (Table 2).

The highest solubility in SGF was unexpected for CMS-Pf because normally CMS is protonated in SGF with a loss of solubility. The high solubility of the complex CMS-Pf is a proof of complexation when carboxylic groups complexed with the Pf alkaloid are unable to protonate in gastric acidity. Generally, the solubility of Pf alone is lower than CMS-Pf complex, approximately 4-5 times in all media. This is another proof that the high increase of solubility is due to the complexation of CMS-Pf.

There are not many natural soluble compounds with a high antioxidant activity. Pf and derivatives (MD/Pf, CMS-Pf) could be used in this regard for several purposes.

TABLE 2
Solubility of various forms in the different media according to the USP-NF
Solubility in various media (mg/mL)

| Sample | Water | SGF | SIF | Description term |
|---|---|---|---|---|
| Pf | 27.32 ± 0.88 | 31.07 ± 0.01 | 24.49 ± 0.38 | Sparingly soluble |
| MD/Pf | 44.87 ± 3.69 | 44.28 ± 2.05 | 38.97 ± 1.68 | Soluble |
| CMS-Pf | 72.55 ± 2.64 | 147.88 ± 8.46* | 87.65 ± 0.95 | Soluble; *Freely |
| | | | | soluble |

With enhanced Pf solubility, it could be easy to formulate dosage forms for controlled release.

Preliminary dissolution tests revealed that complexation of Pf with CMS could prolong for several hours and gradually the release of Pf. This is a major achievement versus the tablet forms of Pf and MD/Pf which release about 700 mg in less than 50 min in the SGF. When Pf was complexed with CMS, the release profile was dependent on the DS of the CMS. Monolithic tablet dosage forms containing about 450 mg of Pf for a sustained release of about 8-14 h were formulate with different free CMS excipients to obtain four different formulations of CMS-Pf complex as active principle (FIG. 12).

In vitro liberation pattern showed that CMS-Pf complex formulations were able to provide a slow release of Pf in simulated gastric (2 h) and then in intestinal fluids a sustained release during 9-12 hours according to the DS of the CMS matrix and to the HPMC binding agent amount (FIG. 12). The Pf sustained release is expected to ensure a long-lasting action.

### 1.4. Conclusion and Perspectives

The Pf, a natural alkaloid with high antioxidant activity was coated by MD or complexed with CMS to increase solubility without modification of its antioxidant activity. The complexation with CMS may generate interactions between amine group (of Pf) and carboxylate group (of CMS). These interactions present several advantages such as enhanced solubility, increased stability of Pf, extended release time of Pf and enhanced bioavailability, as requested for long lasting action. Thus, coated Pf and also complexed Pf are able to prevent undesirable interactions or antagonistic effects when associated to other bioactive ingredients.

For evaluation of solubility specifically for a bioactive agent (i.e. Pf), a faster and simpler approach was proposed to quantify effective solubility of one compound of a sample based on one of its intrinsic properties: i.e absorption at 280 nm and antioxidant capacities of Pf. This approach seems adequate for various active agents, mostly for complexes and mixed compounds.

Because of its good antioxidant activity, the Pf could be associated to several bioactive agents in combined therapy for sustained released formulations as combined therapies against many pathologies, as malaria.

### Example 2

### Complexation of PF with CMC

### 2.1 Synthesis of CMS

CMS were prepared as detailed above in Example 1, and three CMS with DS of 11%, 17% and 24% (0.11, 0.17, and 0.24) were obtained.

### 2.2 Preparation of CMS-Pf and CMC-Pf Complexes

CMS-Pf complexes were prepared as detailed above in Example 1. Briefly, CMS was dispersed at 8.5% in water. After homogenization and obtaining a homogenous suspension, an amount of 50 mL of CMS suspension was slowly added in 450 ml of Pf 21.5% previously dissolved in ethanol (60%). Similar operation was carried out for the CMS and for CMC (high viscosity) with a degree of substitution of 0.8. The final ratio Pf/carboxyl polymer (w/w) was respectively about of 96/4. The solutions were maintained under agitation at room temperature until spray-drying to obtain powders using the following parameters: 1) inlet temperature 150° C.; 2) outlet temperature 100° C.; 3) spray flow 500 L/h, and 4) aspirator setting at 38 m³/h. During the drying process, the polymeric excipient/Pf suspension was continuously gently stirred to prevent sedimentation. The resulting powder was used to prepare monolithic tablets (12.5 mm diameter, 3.0 mm thickness) containing 400 mg of Pf by direct compaction. The kinetics of the drug release were recorded using the Distek™ dissolution (100 rpm) and the detection of Pf was monitored at 280 nm, first in a dissolution medium made of 1 L of SGF (pH 1.5) during 2 h. Then, the tablets were transferred in 1 L of SIF (pH 7.2) at 37° C.) (USP 32 method).

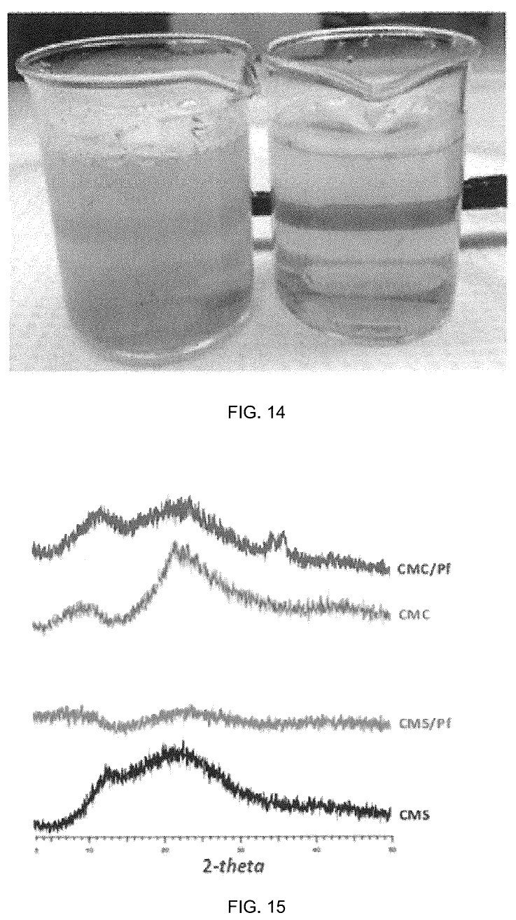

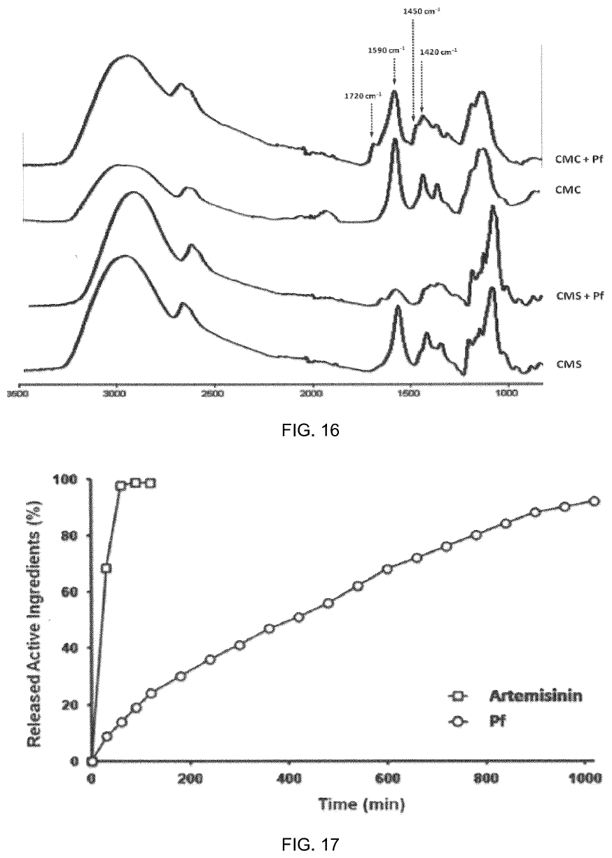

A significant stability after one month was observed for CMS with high degree of substitution (0.24). However, the release of uncomplexed Pf (i.e. Pf alone) was rapid and complete in simulated gastric fluid after 40 min. A similar stability was observed with CMC, but the release of the drug was longer and can reach 2 h in SGF. The tablets of Pf/CMC complex generated a completely clear dissolution medium as compared with that of Pf/CMS complex (FIG. 14), suggesting that probably CMC with 0.8 DS improves the solubility of Pf. It appears that the stability of Pf is dependent of the degree of substitution with the carboxylate groups. At higher DS more carboxylate groups are available to stabilize Pf alkaloids. It is of interest to mention that to obtain starch derivative with higher DS (i.e. up to 0.8 or even 1.0), the reaction must be essentially carried out in non-aqueous media. For the moment, commercial soluble CMC with a DS about of 0.8 seems appropriate to complexate with Pf amine group and to improve the stability of tablets and the release kinetics.

### 2.3 X-Ray Diffraction (X-RD) Analysis

Now referring to FIG. 15. X-RD analysis showed that peaks of CMS (DS 0.24) and CMC (DS 0.8) presented two crystalline levels. However, the peak of CMS at 2-theta=220° is larger and less crystalline than that of CMC. The Pf/CMC complex seems to retain its crystalline property, despite a slight decrease in the intensity. In contrast, the CMS/Pf complex showed a marked loss in its crystallinity and intensity. The difference in the crystallinity may account for the large amount of interactions (an important amount of carboxylate groups which can interact with a greater number of amine groups from Pf) due to the presence of higher DS. This can be explained by the fact that CMC at higher DS that can interact with a greater number of amine groups from Pf increasing the stability of the complex Pf/CMC. With regard to the complex CMS/Pf, the ionic interaction between the carboxylate and amino groups seems lower in comparison with Pf/CMC complex. In this case, the interactions were probably limited to weak hydrogen bonding, explaining thus a rapid release of Pf from the complex in SGF.

### 2.4 FTIR Analysis

CMS absorption bands appeared at 1590 and 1415 cm⁻¹ are assigned to carboxylate anions (asymmetric and symmetric stretching vibrations). Similar stretching vibrations of the carboxylate from CMC were observed.

For CMS/Pf complex, a decrease of the absorption at 1590 cm⁻¹ was observed, probably due to interactions between carboxylate groups of CMS and amine groups from Pf with an absorption band appearing at 1450 cm⁻¹, whereas CMS alone exhibits no significant absorption band related to available carboxylate groups, probably due to the presence of low DS in CMS. Similar profile was observed for Pf/CMC complex. However, the intensity of the absorption band located at 1590 cm⁻¹ ascribed to carboxylate groups is slightly decreased as compared to CMC alone. This phenomenon is probably due to high DS (0.8) in CMC, suggesting that after complexation with Pf, an amount of free carboxylates still remain available. These observations explain why the Pf/CMC complex is more soluble than the initial Pf raw material coated with maltodextrin.

The results above suggest that the Pf can be complexed with up to 3.5% CMC, suitable for good interactions between amine groups of Pf and carboxylate groups of CMC. These interactions present several advantages, such as increased stability of Pf, enhanced solubility and consequently, bioavailability of Pf, extended release time of Pf, as requested for long lasting action, prevented undesirable interactions or antagonistic effects with other bioactive ingredients, particularly, in the present case, with Artemisinin.

### Example 3

### Formulation of Artemisinin/Pf for Combination Therapy

Since the desired ACT pharmaceutical combination requires an immediate release for Artemisinin and a sustained release for Pf, a bi-layer dosage form appeared appropriate for the formulation of the combination therapy.

### 3.1 Bi-Layer Tablet Dosage Form

### 3.1.1 Formulation for the Artemisinin Immediate Release

100 mg of Artemisinin (short half-life) were mixed with Explotab (cross-linked CMS) which can rapidly hydrate and results in the disintegration of the Artemisinin layer, with the immediate release of the bioactive agent.

### 3.1.2 Formulation for Pf Sustained Release

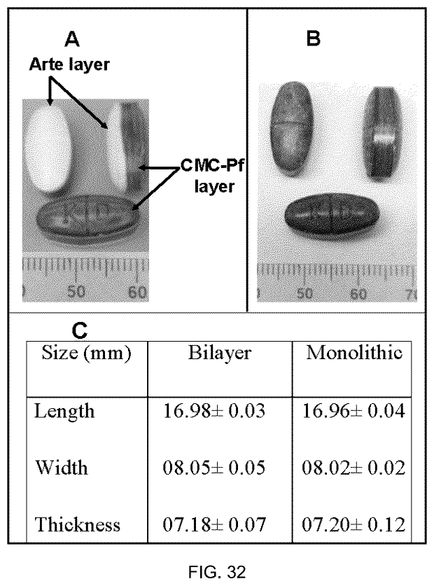

The Pf/CMC complex was prepared as above and the ratio Pf/CMC was approximately of 96/4 (w/w). The preliminary sustained release formulation consisted in 415 mg of Pf/CMC complex powder mixed with 120 mg of CMS (DS 0.24) and 100 mg of hydroxypropyl methylcellulose (HPMC). Bi-layer tablet was obtained first by direct compression of Pf/CMC complex with HMPC at 1.6 T/cm², followed by filling with the mixture of Artemisinin and Explotab™ directly onto the compressed Pf/CMC layer and finally by compacting at 2.3 T/cm² the combination to obtain a bi-layer tablet (FIG. 32).

### 3.1.3 Dissolution Assay

The preliminary release kinetics of Artemisinin/Pf from the combination forms are presented in FIG. 17. Artemisinin is rapidly released and completed after 60 min, whereas Pf is slowly released for a period of about 16 h.

The release kinetics of Artemisinin/Pf bilayer tablet fits well for the requested criteria: Artemisinin possessing fast action and short biological half-life is rapidly released for acute treatment, while Pf slow release is expected to ensure a long lasting action.

### Example 4

### Monolithic Artemisinin/PF Combination Therapy Tablet Dosage Form

Although bi-layer tablet formulation is functional, the manufacturing price of this dosage form is elevated: it requires special equipments and longer operating time due to multistep processing. This will give a product that is not affordable for all people in need. In this context, a monolithic tablet dosage form was developed, which is of lower production cost.

### 4.1 Preparation and Characterization of CMC/Pf Complex

The CMC/Pf complex was prepared as detailed above. Pharmaceutical grade CMC and CMS were used. CMC Degree of Substitution was high (DS 0.7) and comparatively the DS of CMS was moderate (DS 0.2). The CMC/Pf complex was obtained by mixing CMC solution with Pf previously dissolved in ethanol. After homogenization, CMC/Pf complex powders were obtained by spray-drying. The concentration of CMC in the final product was approximately 3.8%.

After dissolving an amount of 400 mg of powders (MD/Pf or CMC/Pf) in SGF or SIF at 37° C. during 30 minutes, the CMC/Pf complex showed a homogenous and clear solution whereas the MD/Pf presented agglomerations with insoluble particles which are bonded each other on the surface of the SGF solution.

### 4.2 FTIR Analysis of Pf Complexes

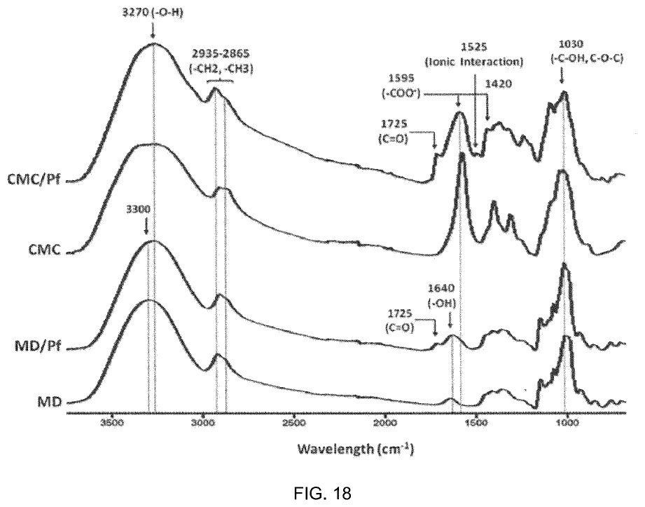

Now referring to FIG. 18. For MD, the IR spectral profile shows stretching vibration at 3300 and 1640 cm⁻¹ assigned to O—H groups, and at 1010 cm⁻¹ for C—O groups. No considerable change for MD/Pf aggregates were noticed, except a new band at 1725 cm⁻¹ attributed to carbonyl groups from Pf. For CMC, the absorption bands at 1590 and 1415 cm⁻¹ are ascribed to asymmetric and symmetric stretching vibrations of carboxylate anions (—COO⁻). A new band at 1725 cm⁻¹ was attributed to carbonyl groups under protonated form.

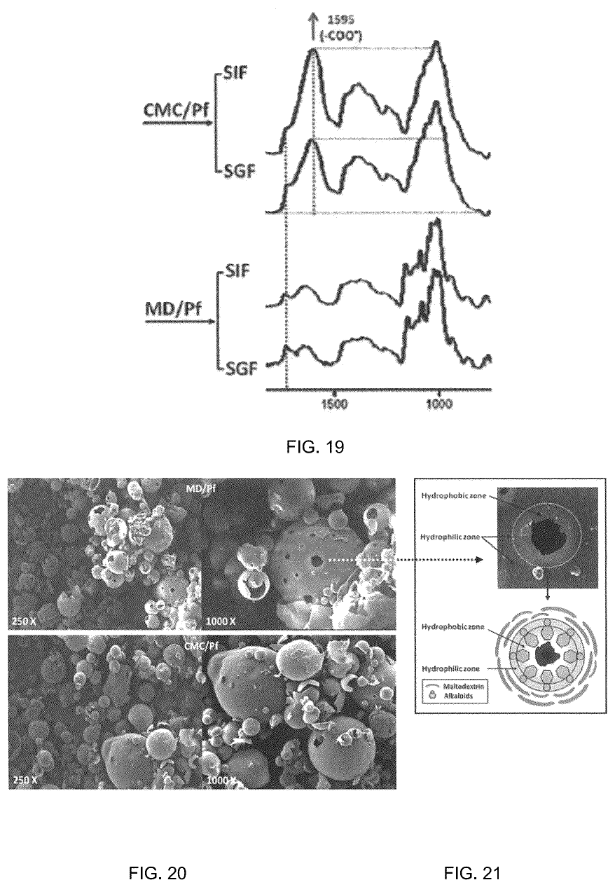

No marked differences (FIG. 19) in the FTIR patterns were found for the MD/Pf aggregate incubated in SGF (2 h) and SIF (2 h). For CMC/Pf complex after incubation in SGF, a reduction in intensity at band at 1595 cm⁻¹ was noticed. This phenomenon may be related to the protonation of carboxylate to carboxylic acid groups and is probably due to the ionic interaction between carboxylate groups from CMC and ionized ammonium groups of Pf. In contrast, when transferred in SIF a re-increase in intensity of carboxylate bands (1595 cm⁻¹ was observed, due to the deprotonation of carboxylic acid group.

### 4.3 X-Ray Diffraction (X-RD) Analysis

X-RD showed that maltodextrin (MD, alone) and Pf coated with maltodextrin (MD/Pf) presented a similar profile with a main band located at an angle 2 theta 21°. After incubation of the MD/Pf aggregate in SGF and in SIF, a new crystalline form appeared with a band at 2 theta 14° whereas the band at 21° remained generally unchanged. The X-ray patterns indicate a more organized and consequently more stable structures of MD/Pf after incubation in SGF and SIF solutions. This stability of MD/Pf explained why MD/Pf is less soluble in SGF and SIF.

In contrast, the CMC/Pf complex presented a different profile. For CMC alone, X-ray diffractogram showed two bands at angle 2 theta 9° and 23°. When complexed as CMC/Pf, its X-ray diffractogram is changed: the band of 2 theta 9° shifted to 12° whereas the band at 23° showed a marked loss in its intensity and crystallinity. These losses may reflect the fact that CMC with higher DS (0.70) possesses considerable interactions (an important amount of carboxylate groups interacted with a greater number of amine groups from Pf).

After incubation of CMC/Pf complex in SGF and SIF, the band at 12° become broader whereas that located at 23° is almost lost and crystallinity reduced, indicating that the CMC/Pf complex is poorly organized with an amorphous structure which can explain its higher solubility in SGF and SIF media.

### 4.4 Scanning Electron Microscopy of the Pf Complexes

SEM (at magnification 250 and 1000×) of Pf aggregated with MD or complexed with CMC showed granules with spherical shape and variable sizes (FIG. 20). However, MD/Pf granules present spherical surfaces with numerous holes whereas the surface of CMC/Pf is smoother with only a few holes. The explanation of this phenomenon is probably related to the size of MD and its interaction with Pf. In fact, MD is an oligosaccharide consisting of D-glucose units with chain lengths from 3 to 17 glucose units. The MD used to coat Pf is a small compound unable to completely cover Pf granules.

Furthermore, the MD is hydrophilic and polar whereas Pf is partially hydrophobic and negatively charged (FIG. 21)—these are opposed characteristics that can generate a repulsive force on certain granule areas, leading to the formation of holes. In CMC/Pf complex, the CMC is a polysaccharide possessing a greater size and long chains that can totally cover the Pf granules. Additionally, its carboxylic negative charges can ionically interact with amine groups of Pf, generating thus stabilization and explaining why CMC/Pf exhibited a smooth surface.

4.5. In Vitro Study of Release Kinetics of Artemisinin Combined with CMC/Pf

### 4.5.1 Monolithic Formulation

The CMC/Pf complex was prepared as mentioned above and the sustained release tablet formulation consisted of 400 mg of CMC/Pf complex powders mixed with 100 mg of artemisinin. As content of monolithic dosage, the excipients CMC (DS 0.7) 200 mg, HPMC 100 mg, croscarmellose™ 10 mg, and magnesium stearate 15 mg were added to the mixture with the active agents. The powder mix was compacted at 2.3 T/cm² to obtain monolithic tablets under biconvex oval shape.

### 4.5.2 Dissolution Media and Methods to Evaluate the Release Kinetics

For each tablet, the dissolution was started in 1 L of SGF (pH 1.5) for 2h and followed in 1 L of SIF (pH 7.2) at 37° C., according to USP 32. The drug release kinetics were recorded using a Distek™ dissolution 2100A paddle system at 100 rpm.

The quantification of Pf was carried out at 280 nm, whereas the dosage of artemisinin was realized by spectrophotometry at 734 nm after filtration and dissolving artemisinin in the acetone/water (ratio 40:60) solvent containing ABTS (2,2′-azino-bis[3-ethylbenzothiazoline-6-sulphonate]).

### 4.5.3 Release Kinetic Profile

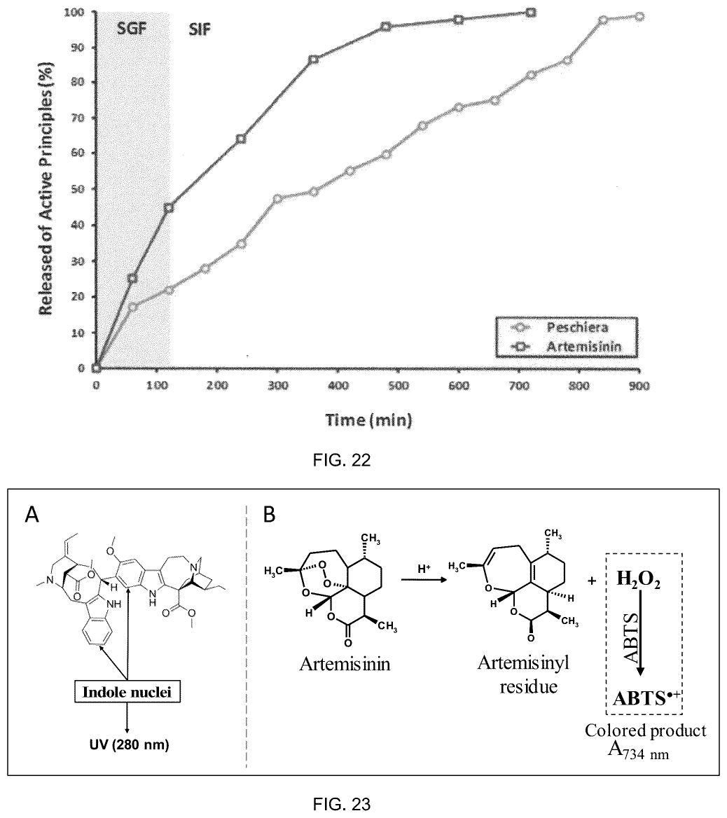

The release kinetics of Art/Pf from monolithic tablet (FIG. 22) showed that about 45% of Art was released after 2 h in SGF and the complete release was achieved after 8 h, whereas Pf was slowly released for a period of about 12 h.

### 4.6.1 In Vitro Study of Artemisinin and Pf Interactions in SGF

Since both bioactive agents, Art and Pf will be used in a combination therapy in the same dosage form, there is a risk of molecular interactions to be considered. These interactions may be beneficial due to the antioxidant properties of Pf, which will reduce the lactone form of Art to a lactol form (Dihydroartemisin) that is more soluble and more effective. These interactions could also cause a moderate loss of activity due to the partial degradation of the endoperoxide bridge of artemisinin. Furthermore, artemisinin could be degraded in the acidic medium leading to ring opening driven by protonation of the endoperoxide group which can subsequently be reduced to 4-hydroxy deoxyartemisinin, deoxyartemisinin or 9,10-dihydroartemisinin, etc. In order to examine the drug-drug interactions as well as their stability in acidic medium, artemisinin and Pf were incubated in simulated gastric fluid (pH 1.5, at 37° C.) during 2 h prior to FTIR and NMR analyses.

### 4.6.1.1 FTIR Analysis

The results showed that there are no significant differences between FTIR spectra (untreated artemisinin, Art alone and ART/Pf in SGF, FIG. 31A). If Art is reduced to lactol form, a new absorption band assigned to hydroxyl group (—OH) should appear at about of 1640 cm⁻¹, or it was not the case in this study.

### 4.6.2.2 1H NMR Analysis

Similar results were observed by ¹H NMR (FIG. 31B) with no marked differences detected. Furthermore, no degradation of artemisinin was observed in SGF. Generally, artemisinin is insoluble in majority of aqueous solvents and consequently stable in gastric acid. Additionally, artemisinin was found to be more stable than its derivatives in the simulated gastric fluid (pH 2.0, 37° C.), probably due its lower solubility.

### Example 5

### Complexation of PF with CMS

5.1 Preparation of Polysaccharide Complexes with Pf

CMS with different degrees of substitution was synthetized from high amylose starch (Hylon VII) as described by Assaad and Mateescu (2010) and the DS was determined by titrimetry (Le Tien et al., 2004).

Briefly, in 250 mL Hylon VII suspension (100 g in distilled water) for hydration for 10 min at 50-60° C., 200 mL of 4 M NaOH was added for gelatinization under continuous stirring for 1 h. Various quantities (60-100 g) of sodium monochloroacetate (solubilized in 120 mL water) were added for 24 h of reaction, under continuous stirring. After neutralization (pH 7.0) of the solution with acetic acid, the CMS was precipitated by adding an excess of ethanol/water (70:30, v/v) solution and was filtered on Whatman cellulose filter paper. Then, the CMS was dehydrated by washing three times with pure acetone and the CMS mass was air-dried for residual solvent elimination and obtaining of the powder.

The Peschiera fuchsiaefolia (Pf) pure extract was complexed respectively with CMC and CMS. Also, it was coated with maltodextrin (MD) to obtain respectively CMC-Pf, CMS-Pf complexes and the MD/Pf coating. For the CMC-Pf and CMS-Pf complexes, they were obtained by mixing carboxyl polysaccharide solution with Pf previously dissolved in ethanol (60%). After homogenization, CMC-Pf or CMS-Pf complex powders were obtained by spray-drying (Büchi Mini Spray Dryer B-290, Büchi Labortechnik AG, Flawil, Switzerland). Practically, the CMC or CMS was dispersed at 1% in water. After homogenization, a volume of 400 mL of CMC suspension was slowly added in 480 ml of Pf 20% dissolved in ethanol 60%. The final ratio Pf/carboxyl polymer (w/w) was respectively about 96/4. These solutions were maintained under agitation at room temperature until spray-drying to obtain CMC-Pf or CMS-Pf complex powder depending of used polymer. The spray-dryer parameters were: i) inlet temperature 150° C.; ii) outlet temperature 100° C.; iii) spray flow 500 L/h, and iv) aspirator setting at 38 m³/h. During the drying process, the Pf/polymeric excipient suspension was gently stirred for avoiding sedimentation. The concentration of CMC or CMS in the final product was approximately 4%. The coated MD/Pf was obtained in the same conditions, but the MD/Pf (w/w) ratio used was 10/90; thus, concentration of MD in MD/Pf coated complex was 10%.

### 5.2 Characterization

### 5.2.1 Fourier Transform Infrared (FTIR) Spectroscopy

For comparative study, different samples (CMC, CMS, CMC-Pf, CMS-Pf and MD/Pf) were analyzed before and after incubation in SGF (pH 1.2) for 2 h and in SIF (pH 6.8) for 2 h.

The spectra (FTIR) of different samples under tablet form (CMC, CMS, MD, CMC-Pf complex, CMS-Pf complex and MD/Pf coating complex) were performed (before and after treatment) in Thermo Scientific Nicolet 6700/Smart iTR (Madison, WI, USA). FTIR spectrometer equipped with a deuterated triglycinesulfate-KBr (DTGS-KBr) detector and a diamond smart attenuated total reflection (ATR) platform. Before taking each spectrum, the crystal was cleaned with ethanol and a background spectrum was acquired. The parameters used in the analyses were: number of scans, 32; resolution, 4 cm⁻¹. All the analyses were carried out at room temperature (25±1° C.).

### 5.2.2 X-Ray Diffraction (X-RD)

The diffraction patterns of CMC, CMC-Pf, MD and MD/Pf were recorded using a Siemens D-5000 diffractometer (Munich, Germany) with a SOL-X detector and a Cobalt cathode in reflectance mode at a wavelength of 1.789 Å. The diffractograms were registered over an angular range of 20 from 0° to 30° and a scan rate of 2° per minute and treated using Diffracplus software.

### 5.2.3 Scanning Electron Microscopy (SEM)

The microstructures of Pf, MD, MD/Pf and CMS-Pf were taken with a JSM-6010LV InTouchScope™ (SEM) (JEOL, Tokyo, Japan) using a secondary electron image detector. Small amount of powder was mounted on a metal stub with an adhesive and covered under vacuum with a fine layer of gold in a sputter coater BIO-RAD E5200 (Bio-Rad Laboratories Ltd., London, UK). The SEM microphotographs were obtained by accelerating voltages of 1.5 kV and at high vacuum.

### 5.2.4 Thermogravimetry Analysis

Thermogravimetric analyses (TGA) were conducted with TA Instruments TGA (Q500)/Discovery MS. Samples of CMC-Pf complex, CMC and Pf (1-10 mg), placed in platinum pans, were heated from 30 to 400° C. at a temperature variation rate of 10° C./min, under a constant nitrogen flow (100 mL/min). Thus, thermogravimetric curves (thermogram) were obtained and the weight loss (%) was calculated.

### 5.3 Stability of CMC-Pf

Structural stability of CMC-Pf was evaluated after incubating in SGF and in SIF. Tablets of CMC-Pf obtained by direct compression of the powders (2.0 T/cm²) under flat-faced round shape, were incubated (0-2h at 37° C.) in SGF and/or in SIF, then dried and the structure was analyzed by FTIR. On the other hand, sample was dissolved and incubated (0-2h at 37° C.) in SGF or SIF before antioxidant activity measurement (method described below).

### 5.4 Antioxidant Properties

Antioxidant activity of CMC-Pf complex was evaluated and compared to CMS-Pf complex and Pf. Samples were dissolved in medium (nanopure water, SGF or SIF) and antioxidant properties evaluated by the improved TEAC (Trolox Equivalent Antioxidant Capacity) fast assay as described by Konan et al., 2016. Indeed, a volume of 6.5 mL (140 μM in 0.85% NaCl) ABTS was electrolyzed (400 Volts, 10 mA) for 20 seconds to generate the ABTS•+ radical. The two electrodes were in platinum, with 2.12 mm diameter, 25.33 mm length and inter-electrode space of 26.36 mm measured with an Electronic Digital Caliper (Docap Corp., St-Laurent, Quebec, Canada) for the range 0-200 mm with readings at a precision of tens of microns.

For the analysis, a volume of 950 μL of ABTS^{•+} solution was mixed with 50 μL of 0-200 mM Trolox (standard curve) or 50 μL of the tested samples (Pf, MD/Pf or CMC-Pf) and the absorbance at 734 nm of the initial mixture with the electrolysis-induced ABTS•+ was measured after 1 min. The decrease of absorbency due to the antioxidant capacity reflects the scavenging of the free radical (ABTS^{•+}). The ability of Pf, MD/Pf and Pf-CMS to scavenge the ABTS^{•+} radical cations were compared to that of Trolox (structural hydrosoluble analogue of vitamin E) used as reference antioxidant. The Trolox equivalence corresponds to the Trolox concentration having the same activity as the tested sample at a given concentration. The results are expressed in μM or mM of Trolox equivalence per mass (mg) of product.

Each measurement consisted of at least five readings and the values represented the mean±standard deviations.

### 5.5 Solubility Assay

Solubility of CMC-Pf in different media (nanopure water, Simulated Gastric Fluid [SGF], Simulated Intestinal Fluid [SIF]) was evaluated as described by Konan et al., (2018) approach. The solubility value (mg/mL) which is the maximal concentration (mg/ml) of sample in a medium after saturation was determined on basis of absorbency at 280 nm. An excess of CMC-Pf (approx. 2 g) in 10 mL of the medium (nanopure water, SGF or SIF) was shaken at 37° C. for 5 h and the mixture was centrifuged at 2000×g for 10 min. The supernatant was filtered with 0.45 μm Millipore filter. The effective concentration (mg/mL) of Pf in an aliquot (1 mL) was evaluated in UV at 280 nm (described in dissolution assay section) as a function of sample concentration (mg/mL) using a standard curve previously established.

### 5.6 Dosage Methods of Pf and Arte

### 5.6.1 Determination of Maximal Absorption Values of Pf and CMC-Pf Complex

The UV-visible absorption spectra of Pf and CMC-Pf complex filtered solutions were acquired using a UV-Visible spectrometer (Genesys™ 10S UV-Vis, Thermo Scientific, USA) in the range of 190-700 nm. The maximum absorbency is 280 nm.

5.6.2 Quantitation of Pf Released from Dissolution Assay

Quantitation of Pf was done by UV-Vis spectrometry at 280 nm (FIG. 23A), based on indole nuclei of alkaloids (active agents) of Pf extract. An aliquot (1 mL) of released solution was filtered (pore diameter 0.45 μm) and the absorbance at 280 nm of 1 mL was read.

5.6.3 Quantitation of Artemisinin Released from Dissolution Assay

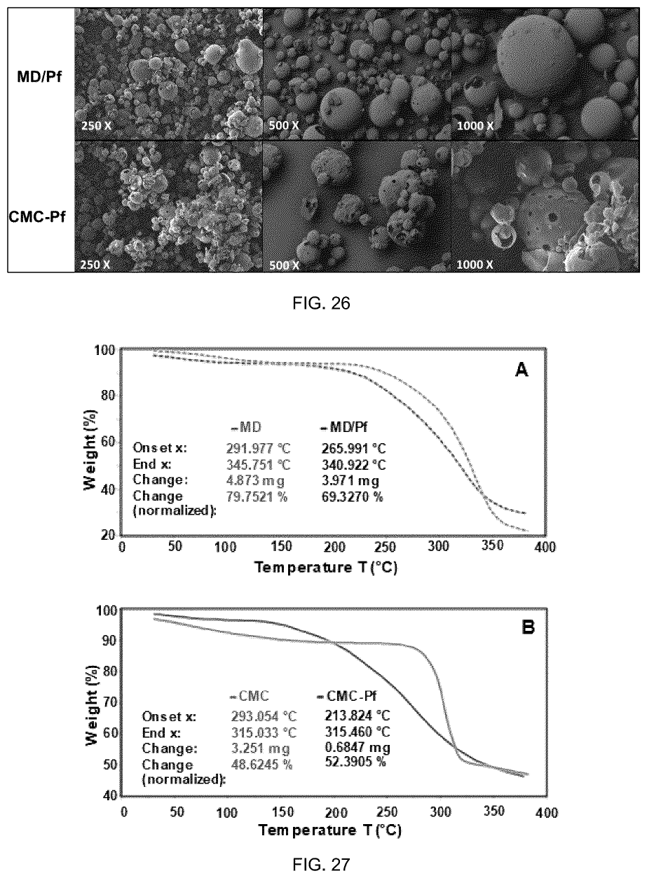

For Arte, a spectrophotometric method was used. It consisted in using the chromogenic ABTS (initially colorless) reagent that was oxidized by the endoperoxide of Artemisinin in acidic medium to form a radical cation (ABTS•+, colored, FIG. 26B). The generated ABTS•+ color (measured at 734 nm) is directly proportional to the concentration of Artemisinin present in the medium. An amount of 1% sulfuric acid was added (in excess) in order to break the endoperoxide bond and liberate hydrogen peroxide (H₂O₂). This H₂O₂ reacted with ABTS reagent and developed a new colored product, the ABTS^{•+} (FIG. 23B) with an intensity proportional to the quantity of Artemisinin present in the sample.

Standard curves previously established in appropriate medium (nanopure water, SGF or SIF) permitted to determine the quantity of Pf and of Arte.

### 5.7 In Vitro Evaluation of Arte and Pf Interaction

In order to examine the drug-drug interactions as well as their stabilities in gastric acidity, Artemisinin and Pf were incubated in SGF (pH 1.5, at 37° C.) for 2 h and analyzed by FTIR, NMR and antioxidant capacity.

After incubation, Arte was separated from Pf by decantation, washed and dried before FTIR and RMN analyses; whereas the Pf solution (in presence or not of Arte) was filtered and the antioxidant capacity was evaluated as previously described in the section of antioxidant properties.

5.8 Formulation of Arte Combined with CMC-Pf Tablets

The one factor at a time (OFAT) approach was used to optimize the formulation and achieving the desired release percentage. This consists in the variation of one factor at a time and keep the other factors constant. The quantities of Arte and CMC-Pf (bioactive agent) were fixed, respectively 100 mg and 450 mg. Bilayer and monolithic tablet dosage forms were formulated. A minimum of 50 tablets were prepared for each batch.

### 5.8.1 Bilayer Tablet Formulation

The Arte layer consisted in mixing of 100 mg of Arte with cross-linked sodium cellulose (Croscarmellose sodium, XC, 2-150 mg). The second layer was composed of 400 mg of CMC-Pf complex powder mixed with 120 mg of CMC (DS 80%), 100 mg of hydroxypropyl methylcellulose (HPMC) and 15 mg of magnesium stearate. The bilayer tablet was obtained first by direct compression (Carver Press, Wabash, IN, USA) of CMC-Pf complex mixed powders at 1.3 T/cm², followed by filling the Artemisinin and XC mixed powders directly on the CMC-Pf layer and finally compaction at 2.3 T/cm² to obtain bilayer tablet dosage form. Size of tablets (length, width and thickness) measured with an Electronic Digital Caliper (Docap Corp., St-Laurent, Quebec, Canada).

### 5.8.2 Monolithic Tablet Formulation

Monolithic tablet dosage form contained 100 mg of Arte, 10 mg of XC, 400 mg of Pf, 100 mg of HPMC, 200 mg of CMC and 15 mg of magnesium stearate. These dry powders were directly compacted in the flat-faced punches to obtain the monolithic tablet.

### 5.9 Disintegration Test

Approximate disintegration time of tablets was determined using an ED-2L disintegrator tester (Electrolab, India) as per USP 40/701 specifications. Tablets (n=6) were placed in each basket of the disintegration test apparatus. The time required for disintegration was measured at 37±2° C. at 25 rpm using first 900 mL SGF during one hour, then 900 mL SIF for total disintegration. The tablet was supposed to have disintegrated when all powder particles passed through basket and the time was recorded.

### 5.10 Dissolution Assay

Dissolution assay was done first in simulated gastric fluid (SGF, pH 1.5) for 2 h and then in simulated intestinal fluid (SIF, pH 6.8) as referred by USP method 40 (2017a). Release kinetics from different formulations were followed at 100 rpm and 37° C. using a Distek dissolution system 2100A (Betatek Inc., Markham, ON, Canada) with a Distek TCS 0200 (North Brunswick, NJ, USA). At predetermined intervals (0.5, 1, 2, 4, 6, 8 and10 h), released Pf and Arte were quantified using the appropriate method (described above).

For Pf dosage, a volume (5 mL) was withdrawn from dissolution media, properly filtered (0.45 μm) and diluted before absorbance measurement at 280 nm. To quantify released Arte, the insoluble precipitate was recuperated, washed several times to remove all Pf residues and quantified as previously described.

### 5.11 Mechanic Properties of the Tablets

### 5.11.1 Weight Uniformity Test

The uniformity of tablet weight according USP, was evaluated by randomly

selecting 20 tablets and calculating the average weight; then the tablet weight limits (superior and inferior) were calculated based on the % error of tablets (which is function the weight of tablet):

Superior limit of weight=Mean Weight+(Mean Weight×% error)

Inferior limit of weight=Mean Weight−(Mean Weight×% error)

Because a tablet weight was superior to 324 mg, the % error=±5% following the USP criteria. Tablets used for different tests were those that have a weight in the range of inferior limit to superior limit.

### 5.11.2 Friability Test

Tablets friability was determined based on USP/1216 using Friability Tester (Varian, Inc., Cary, NC, USA). Since the weight of one tablet was superior to 650 mg, ten (10) tablets were used. After cleaning, without dust, the tablets were weighed together, placed in the friabilator drum and were subjected to 100 revolutions at 25 rpm. The tablets were collected, cleaned, reweighed and their relative weight loss percent (% Friability) was calculated as:

$$\% F = ( 1 - W / {W}_{o} ) \times 100$$

Where Wₒ is the weight of tablets before the test and W is the weight of tablets after the test.

### 5.11.3 Hardness Assay

The hardness test was realized according USP/1217. The Tensile strength (T) of tablets was calculated using the following formula:

$$T = 2 F / π \times d \times t$$

F, d and t represented the crushing strength, the diameter and the thickness of tablet, respectively.

The crushing strength F (the force required to break a tablet in a diametric compression was measured using a VK 200 tablet hardness tester (Varian, Inc., Cary, NC, USA). Diameter and thickness were assessed using an Electronic Digital Caliper. The F was expressed in kilopond Kp (according to the hardness tester used) which is equivalent to Kg force.

Because tablets were oval, the crushing strength was applied to the tablets by taking the length or width of tablet as diameter. In each case (diameter as length L or width w), five tablets with diameter and thickness known were used. The force required to break the tablet during a diametric compression was recorded.

### 5.12 Statistical Analysis

All experiments were conducted at least in triplicate and data were presented as the mean±standard deviation.

### 5.13 Characterization of MD/Pf and CMC-Pf Complexes and Stability in SGF and SIF

### 5.13.1 FTIR Analysis

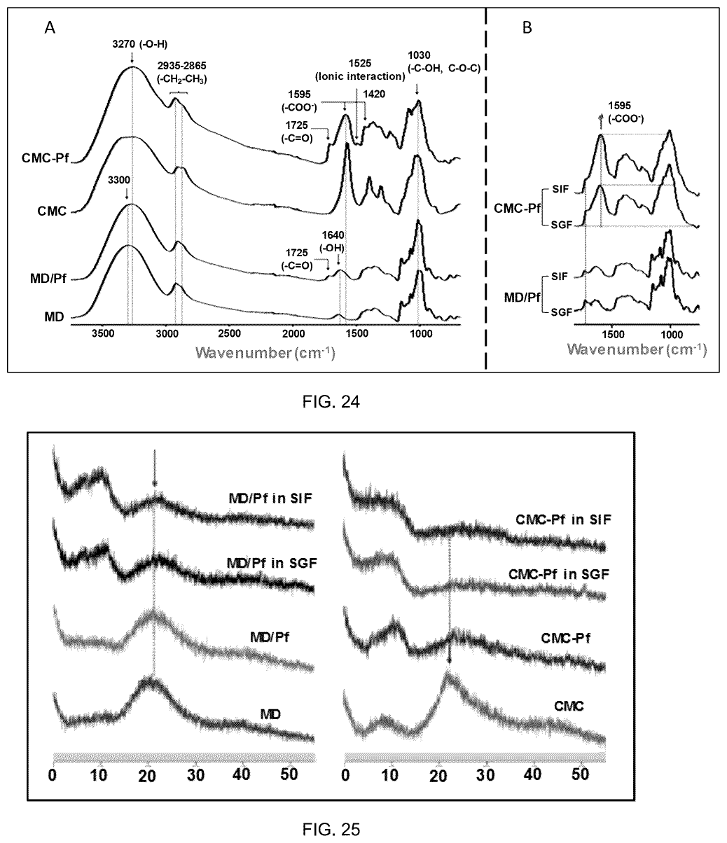

The IR spectral profile of MD showed absorption bands mainly at 3300, 1640 and 1010 cm⁻¹. The MD/Pf coated complex presented similar profile without considerable change, excepted a new band at 1725 cm⁻¹ (FIG. 24A). Similar observation was noticed for CMC-Pf complex

After incubation in SGF (2 h) and SIF (2 h), no marked differences in the FTIR patterns were found for MD/Pf (FIG. 24B). For CMC-Pf complex after incubation in SGF, an intensity reduction of the band at 1595 cm⁻¹ was remarked. In contrast, in SIF, its intensity was re-increased (FIG. 24B).

### 5.13.2 X-RD Analysis

X-ray diffractograms (FIG. 25) showed that maltodextrin (MD, alone) and Pf coated with maltodextrin (MD/Pf) presented a similar profile with a main band located at an angle 2 theta 21°. After incubation of the MD/Pf aggregate in SGF and then in SIF, a new crystalline form appeared with a band at 14° whereas the band at 21° remained generally unchanged (FIG. 25).

In contrast, the CMC and CMC-Pf complex presented a different profile. For CMC alone, X-ray diffractogram showed two bands at 9° and 23°. The main band at 23° indicates that CMC possesses a higher crystalline structure. When complexed with Pf, its X-ray diffractogram is changed: the band at 9° shifted to 12° whereas the band at 23° showed a wide shoulder with marked loss in its intensity and crystallinity. These changes may explain why the CMC-Pf complex is more soluble. After incubation of CMC-Pf complex in SGF and SIF, the band at 12° became broader whereas the one located at 23° was almost lost (FIG. 25).

### 5.13.3 Scanning Electron Microscopy (SEM)

SEM (at magnification 250, 500 and 1000×) of MD/Pf coating complex and CMC-Pf complex showed granules with spherical shape and variable sizes (FIG. 26). However, MD/Pf granules presented spherical smooth surfaces with only a few holes whereas the surface of CMC/Pf exposed numerous holes (FIG. 26).

### 5.13.4 Thermogravimetry Analysis (TGA)

The thermograms (TGA curves) of MD, CMC, MD/Pf coating complex and CMC-Pf complex are presented in FIG. 27. For all samples, a major weight loss from 210° C. was observed.

MD and MD/Pf thermograms generally presented similar profiles. Differently, it was a marked difference for CMC and CMC-Pf thermograms. Indeed, CMC thermogram presented a thermal stability reached approximately at a temperature 300° C. whereas CMC-Pf complex was less stable, with a degradation started from 150° C.

### 5.14 Antioxidant Capacity

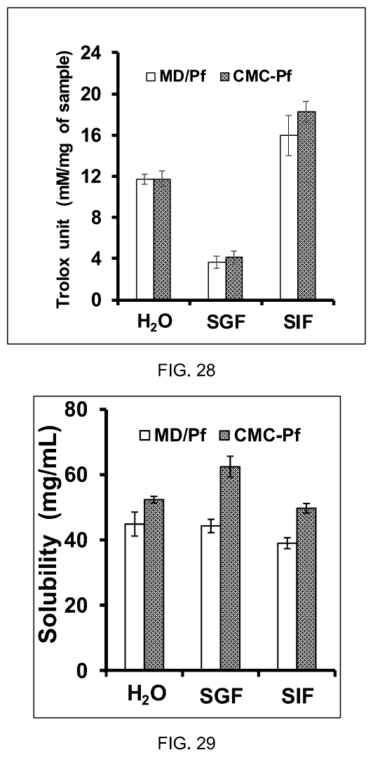

Antioxidant capacity values were expressed in mM Trolox Equivalent units per mg of Pf derivative. No major differences between MD/Pf and CMC-Pf were noticed, but these values depended on simulated gastric or intestinal fluids (FIG. 28). The antioxidant activity was more expressed in SIF and then in nanopure water. In SGF (pH 1.5), the antioxidant activity decreased considerably, about one quarter of that in SIF (pH 6.8) and a third of values obtained in nanopure water (FIG. 28). For MD/Pf, antioxidant capacity values were 11.72±0.49, 3.66±0.61 and 15.94±1.90 Trolox Unit/mg of sample respectively in nanopure water, in SGF and in SIF versus 11.74±0.71, 4.12±0.65 and 18.29±0.97 Trolox Unit/mg of sample for CMC-Pf (FIG. 28).

### 5.14 Solubility Assay

Better solubility was observed for CMC-Pf in different media (FIG. 29). Those are 52.35±0.99, 62.49±3.17 and 49.72±1.43 mg/mL respectively in nanopure water, in SGF and in SIF.

### 5.15 Dosage Methods of Pf and Arte

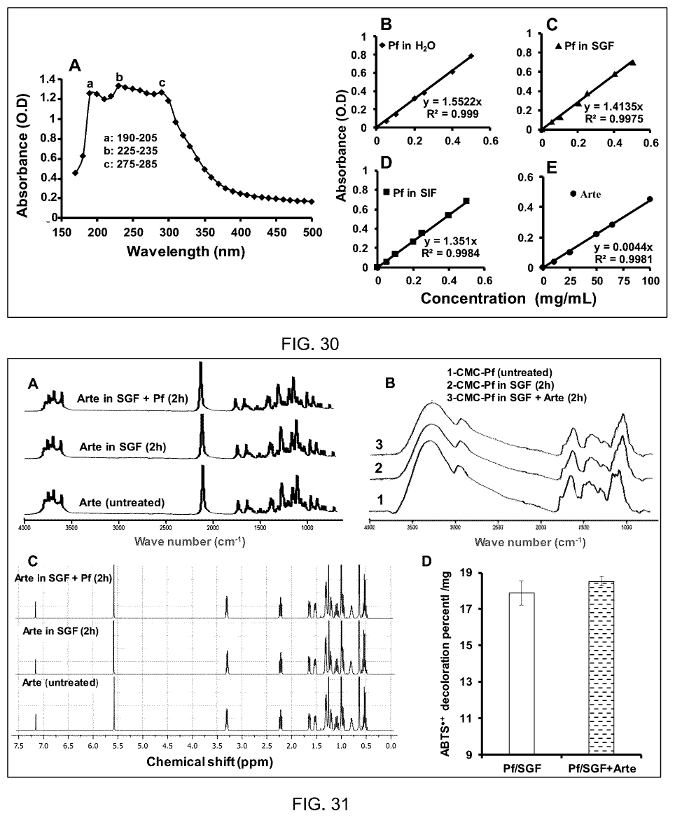

The UV-Vis absorption spectrum of CMC-Pf showed 3 maximal absorption bands at 200 nm, 230 nm and 280 nm (FIG. 30A). The standard curves of Pf (under CMC-Pf form) in nanopure water (FIG. 30B), in SGF (FIG. 30C) and in SIF (FIG. 30D) presented good linearity, as well as that of Arte (FIG. 30E) for quantification methods used. For Pf, quantification was done at 280 nm, whereas the dosage of Arte was realized by spectrophotometry at 734 nm after filtration and dissolving Arte in acetone/water (ratio 40:60) solvent containing ABTS (2,2′-azino-bis[3-ethylbenzothiazoline-6-sulphonate] acid) as described in material and methods section.

### 5.16 Interaction Study Between Arte and CMC-Pf

In vitro interaction of Arte with CMC-Pf was evaluated in SGF. Arte and CMC-Pf were incubated in SGF (pH 1.5, at 37° C.) separately or one in the presence of each other (FIG. 31).

The FTIR analysis showed no significant differences between untreated Arte, Arte alone incubated in SGF and Arte incubated with CMC-Pf in SGF (FIG. 31A). Similarly, no significant difference was observed between CMC-Pf incubated in SGF alone or in the presence of Arte FIG. 31B). Effect of SGF on CMC-Pf was previously showed (FIG. 24B).

Furthermore, the 1H NMR analysis of Arte showed no marked differences between the powders of Arte non-incubated, Arte alone incubated in SGF and Arte incubated with CMC-Pf in SGF (FIG. 31C). Moreover, Arte did not influence the antioxidant property of CMC-Pf (FIG. 31D).

5.17 In Vitro Study of Release Kinetics of Arte Combined with CMC-Pf

### 5.17.1 Aspect and Size of Tablets

The Tablets were formulated under bilayer and monolithic dosage forms (FIG. 32). The bilayer tablet (FIG. 32A) were compressed separately to obtain an Arte layer and a CMC-Pf layer, whereas monolithic tablets (FIG. 32B) contained Arte homogeneously mixed with CMC-Pf and the used excipients.

The sizes of dosage forms are presented in the FIG. 32C (length, width and thickness values of the bilayer and monolithic tablets).

The Table 3 presents the disintegration times according to the type of tablets.

TABLE 3
Type of tablet and disintegration time

| | Disintegration time | |
|---|---|---|
| Tablets | (hours) | Observations |
| Bilayer: | (Arte) 0.17 ± 0.03 | In SGF |
| | (Pf) 10.25 ± 1.2 | Arte layer: total disintegration |
| | | Pf layer: no evidence of |
| | | disintegration, no capping, |
| | | no fissuring. |
| | | In SIF |
| | | Pf layer: total disintegration |
| Monolithic | 10.50 ± 1.5 | In SGF: no evidence of |
| | | disintegration, no capping, |
| | | no fissuring. |
| | | In SIF: total disintegration |

### 5.17.2 Release Kinetic Profile

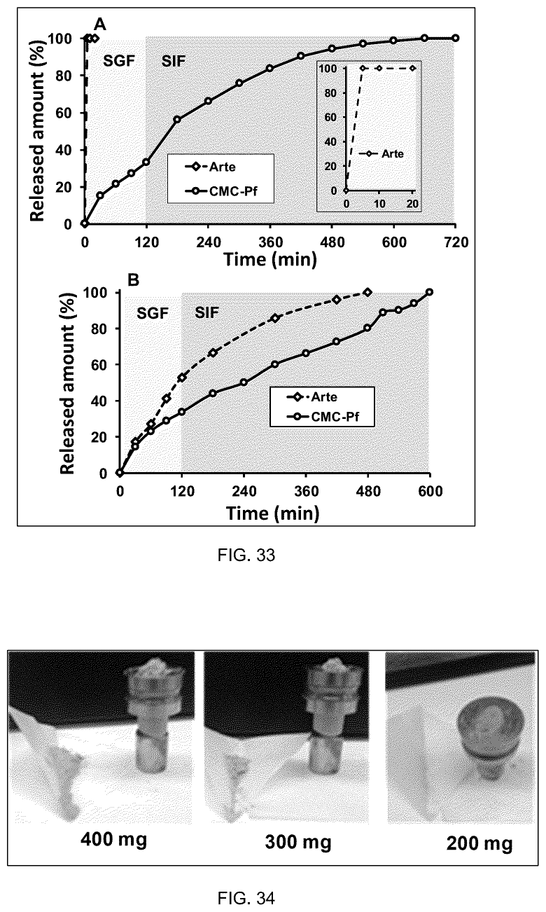

From bilayer tablet, the Arte layer was immediately released (less than 10 min) in the SGF (FIG. 33A), whereas CMC-Pf was slowly released during about 10-12 hours (FIG. 33A). The release kinetics of Arte from combined therapeutic monolithic tablet (FIG. 33B) was about 51% of Arte after 2 h in SGF and the complete release was achieved in SIF after 8 h; whereas the CMC-Pf was slower for about 10-12 h (FIG. 33B).

### 5.18 Mechanical Properties

The Table 4 groups mechanical properties of the two tablet dosage forms (bilayer and monolithic). They are the weight uniformity, friability and the hardness evaluated by the crushing and tensile strengths (Table 4).

TABLE 4
Mechanical properties of the tablets-Length (l) and width (w) were taken
as diameter (dₗ = l or d_{w} = w) for applying of the crushing strength.

| | | | Hardness | | | |
|---|---|---|---|---|---|---|
| | Weight uniformity | Friability | crushing strength | | Tensile strength, T | |
| Tablet form | (interval, mg) | (% mass) | (Kg) | | (Kg/cm²) | |
| Bilayer | [780.60; 862.77] | 0.756 ± 0.00 | dₗ = l | 35.00 ± 1.25 | dₗ = l | 1.80 ± 0.05 |
| | | | d_{w} = w | 35.00 ± 1.33 | d_{w} = w | 1.83 ± 0.02 |
| Monolithic | [771.27; 852.45] | 0.622 ± 0.01 | dₗ = l | 34.58 ± 0.94 | dₗ = l | 0.086 ± 0.00 |
| | | | d_{w} = w | 30.39 ± 2.14 | d_{w} = w | 0.111 ± 0.01 |

### 5.19 Discussion

Peschiera fuchsiaefolia (Pf) is currently commercialized under MD/Pf coated form for reasons such as storage stability and enhanced solubility. Although MD/Pf is soluble in water, but it is poorly soluble in SGF and then in SIF. In contrast, CMC-Pf complex is soluble not only in water, but also in simulated physiological media (SGF and SIF). Additionally, CMC-Pf complex presented several advantages such as low cost, easy to manufacture and more stable during storage.

The CMC derivative is already approved by FDA and currently available in the market with desired DS (0.7-0.8) which favor complexation between the carboxylate and the amine groups of Pf.

In the FTIR spectrum of MD, absorption bands at 3300 and 1640 cm⁻¹ were assigned to O—H groups, and that at 1010 cm⁻¹ to C—O groups (FIG. 24A). With coated MD/Pf, the new absorption band at 1725 cm⁻¹ was attributed to carbonyl group (C═O) from Pf (FIG. 24A).

For FTIR spectrum of CMC, absorption bands at 1590 and 1415 cm⁻¹ were ascribed to asymmetric and symmetric stretching vibrations of carboxylate anions (—COO⁻). After complexation with Pf, a new band at 1725 cm⁻¹ was attributed to carboxylic (—COOH) and a band appeared at 1525 cm⁻¹ (FIG. 24A) was ascribed to the ionic interaction between carboxylate groups from CMC and amine groups of Pf.

After incubation of CMC-Pf in SGF (FIG. 24B), the reduction in intensity of the absorption band may be related mainly to the protonation of carboxylate to carboxylic acid groups (Assaad and Mateescu, 2010). After transfer and incubation in SIF, a re-increase in intensity would be due to the deprotonation of carboxylic acid group ( ) in carboxylate groups (—COO⁻).

The X-ray patterns (FIG. 25) indicated an organized and more stable structure of MD/Pf after incubation in SGF and SIF solutions. This stability of MD/Pf may explain its poor solubility in different media compared to CMC-Pf.

The loss in intensity of the X-ray band at angle 2θ32 23° (FIG. 25) means a reducing of CMC-Pf crystallinity. These suggest that complexes adopted a structure less crystalline (amorphous) which could enhance its solubility in SGF and SIF (FIG. 25).

The SEM analysis is consistent with this above assertion (FIG. 26). During complexation between CMC (carboxylate group) and Pf (amine group), certain repulsive forces would reduce crystallinity on CMC-Pf surface (FIG. 26), resulting an amorphous structure. In fact, CMC is a polysaccharide composed of D-glucose units, of them carrying carboxylate groups. CMC is also hydrophilic and polar whereas Pf is partially hydrophobic (FIG. 21) and low polar feature. Opposite hydrophilic/hydrophobic characteristics can generate repulsive forces on certain granule areas, leading possibly to the formation of holes (FIG. 21).

Maltodextrins possess good film forming properties and used to coat Pf to increase its solubility. Due to its small size (3-20 units of glucose), it was able to cover only partially the Pf granules with less or no repulsive forces generating thus a good stabilization and explaining why coated MD/Pf exhibited a smooth surface (FIG. 26).

Under heating from 25 to 400° C., the TGA minor weight loss (shown by the first stage) corresponded to the desorption of water from samples (loss of intramolecular and intermolecular water) around 50-110° C. The second stage relates to the decomposition or deterioration or degradation (Li et al., 2010; Zhang et al., 2014). All samples were decomposed (deteriorated) between 200 and 350° C. The maximum deterioration temperatures (MDT) were respectively 332.8±2.7° C., 299.26±2.26° C., 320.43±4.19° C., 280.51±8.71° C. for MD, CMC, MD/Pf and Pf-CMC.

Antioxidant capacity showed that Pf forms (MD/Pf and CMC-Pf) are good antioxidants compounds (FIG. 31). Results were expressed as mM of Trolox per mg of sample (Trolox mM/mg of sample). These antioxidant activity values were higher compared to those of several compounds, polyphenol, fruits, plant extract, wine and ceruloplasmin. Difference were high and about 5.10³ to 10⁴ times.

Complexation of Pf with CMC enhanced solubility in different media (FIG. 29), and consequently the bioavailability. According to Pharmacopeia and US National Formulary Substances classification, coated MD/Pf and CMC-Pf complex may be considered as soluble (33 mg/mL to 100 mg/mL) in basis on their solubility (maximal concentration value: volume of water, SGF or SIF required for one part of Pf powder [mg/mL]). Thus, Pf particularly under CMC-Pf complex form, was revealed as one of rare natural soluble compounds possessing high antioxidant activity. It could be used for several purposes.

The three peaks at wavelength of 200, 230 and 280 nm from UV-Vis absorption spectrum of CMC-Pf (FIG. 30A) could be used for Pf dosage. However, the readings at wavelength in region 200-230 nm could present interferences with the carbonyle (C═O) from the matrix. Consequently, the wavelength range 280 nm was selected for spectrophotometric quantification of Pf. The dosage of Arte was carried out by spectrophotometry at 734 nm based on the oxidized ABTS forming the blue-green ABTS^{•+} radical (λ=734 nm) with the hydrogen peroxide liberated from Arte in the presence of strong acid. Good linearities and regression values close to one (FIGS. 30B-E) permitted to the dosage of Pf and of CMC-Pf and CMS-Pf complexes (Konan et al., 2016).

The Arte with an endoperoxide can be altered in gastric acidity leading to ring opening driven by protonation of the endoperoxide group which can subsequently be reduced to deoxyartemisinin or to 9,10-dihydroartemisinin (Haynes et al., 1999). In order to examine the drug-drug interactions as well as their stabilities in gastric acidity, Arte and CMC-Pf were incubated in SGF (pH 1.5, at 37° C.) for 2 h (FIG. 31). No degradation of Arte was observed in SGF in the presence of CMC-Pf neither of CMC-Pf in the presence of Arte (FIG. 31A-C). Furthermore, antioxidant activity of CMC-Pf was not affected by the presence of Arte (FIG. 31D). Generally, Arte is insoluble in majority of aqueous solvents, consequently stable in gastric acid. It was found to be more stable than its derivatives in the simulated gastric fluid (pH 1.5, 37° C.), probably due its lower solubility. Thus, no in vitro interaction between Arte and CMC-Pf was detected.

For release kinetic profiles (FIG. 33), two approaches were explored. The first consisted in a sequential kinetic release: an immediate release for Arte (fast action) and a sustained release for Pf as CMC-Pf complex (long lasting action). Such formulation for the combination therapy was obtained by a bilayer tablet dosage form (FIGS. 32 and 33A). In this formulation, Arte possessing fast action and short biological half-life was rapidly released for acute treatment, while Pf slow release was expected to ensure an action for a long period of time. Then, since the required pharmaceutical combination was of total and immediate release for Arte (≤30 min) in SGF with a sustained release for Pf, only Pf release was quantified by withdrawal of samples at different times (0.5. 1, 2, 4, 6, 8 and 10 h). Arte layer was totally released in the first 30 min avoiding the interference in dosage. Although bilayer tablet formulation fits well with the desired criteria of malaria combined therapy, the manufacturing of this dosage form is currently high cost and complicated. Furthermore, it requires special equipment due to multistep processing and longer operating time. For this reason, it is preferable to develop a monolithic tablet dosage form (FIG. 32B). Its production is simply by compaction of API and excipient powder mixture. The monolithic formulation was conceived to allow a prolonged and simultaneous release of the two active agents at different rates. The liberation pattern was a sustained release of about 10-12 h for the monolithic tablet containing the two active ingredients (Arte combined with CMC-Pf) and using CMC as matrix (FIGS. 32 and 33B). The two bioactive agents would act concomitantly with different target on the parasite. Arte (associated drug) would benefit by inhibition of P-glycoprotein from bisindolic alkaloids of Pf. In fact, an analog of P-glycoprotein is product by Plasmodium to protect itself against any treatment. The inhibitory action of P-glycoprotein by Pf would favor the bioavailability of drugs (Arte and Pf) and probably extend their half-life, increasing thus their antiplasmodial activity, especially for resistant strains. Mechanism of alkaloids from Pf extract would be conducted by alkylation of proteins or a change in pathogen agent membrane permeability, causing cell death. Such formulations will contribute to improve the bioavailability, minimize the drug accumulation at long-term use, reduce fluctuations in drug level, enhance safety by decreasing side effects and increase patient compliance.

All tablets used for different test had uniform weights (Table 4) for each formulation (bilayer and monolithic). The results of each mechanical test are therefore consistent and conform to USP standards (Podczeck, 2012).

The hardness was evaluated using as diameter either the length and the width because the shape of tablets was oval. The high crushing and tensile strengths (Table 4) showed tablets with good mechanical properties and more resistance to capping, fracture and abrasion. This revealed the ability of formulated tablets to withstand the stress of packaging, transportation, handling. Additional, friability percents of tablets from different formulations (Table 4) were lower than 1%, the limit of maximum allowable loss (USP, 1995; Podczeck, 2012) which are conforms with USP norms. To confirm, an in vivo study of CMC-Pf/Arte combination is necessary to highlight its availability in comparing with MD/Pf and its effectiveness comparing with the conventional combination and controlled released interest for antimalarial strategy.

### Conclusion and Perspective

Controlled release anti-malarial based on ACT was formulated by combination of Arte and Pf extract (natural antimalarial agent extracted from plant) previously complexed with CMC as CMC-Pf). These sequential and differential release formulations ensured a long duration of antiplasmodial activity, as requested for long lasting action. It was possible to complex CMC with Pf, generating interactions between amine (Pf) and carboxylate (CMC) resulting CMC-Pf complex. These interactions allowed to enhance solubility, consequently bioavailability, to increase stability in SGF and to prevent undesirable interactions or antagonistic effects with other bioactive ingredients, particularly, in our case, with Arte. Pf biological activity, principally antioxidant activity was preserved after complexation.

### Example 6

### Precompression of Peschiera fuchsiaefolia Powdered Materials to Optimize Density and Compactness

Plant extracts are currently exploited as powder forms of bioactive agents to formulate tablet dosage forms. These dry extracts are often processed using multi-compression press for continuous manufacture of tablets. However, a large majority of plant dry extract doesn't possess initially the required characteristics. In general, dry plant extracts are complex, light, fluffy, non-homogeneous and have low density and poor physico-mechanical properties. Consequently, a pre-treatment may be necessary prior to direct compression. This is as example, the case of Peschiera fuchsiaefolia (Pf) extract, obtained from bark of the plant. As previously described, for better stability and pharmacokinetic properties, the Pf extract was complexed with carboxymethylcellulose (CMC) by spray-drying processus to form the CMC-Pf complex powder. This spray-dried plant extract was formulated in Artemisinin-based Combination Therapy (ACTs) for efficient treatment against Plasmodium falciparum malaria, according WHO recommendation. The manufacturing of tablets at micro-pilot scale with punches for multi-compression may be a major issue because the CMC-Pf light and fluffy character of powder could cause high wall friction properties, remoteness of the particles from each other and occupation of high volume of container (FIG. 34). These phenomena result in a low density (g/mL) and reduced flowability of CMC-Pf. These properties are important in handling and processing operations and for a continuous manufacturing tablets.

Generally, tableting material (active principles, excipients) for dry direct compression into a solid dosage form, must produce good tablet surfaces and strength, particularly in case of automatic tableting equipment. The direct compression vehicle must be compressible, flowable and contained into the dies even at high speed tableting machines. Since the dosage of active material is related to the weight of the tablet, mass variations caused by improper flow must be avoided. The CMC-Pf powders may cause several drawbacks for the production due to: i) low density and poor flowability; ii) hard to fill completely the mixing powders in the die (FIG. 34); and iii) difficulties for automatic press compaction to obtain tablets.

This study was aimed to physically densify the CMC-Pf powder but with no influence on the complexation bond, stability, biological and pharmacokinetic properties. Precompression of the spray-dried plant extract was hypothesized to ameliorate flowability and to improve the compaction processing. It is a pre-treatment of the powder by a mechanic procedure before further use in order to bring particle closer, reduce the occupied volume and so increase the density and flowability. To improve processing, it was important to determine the routine compression force (suitably adjusted) to densify the CMC-Pf complex powders. After precompression of the CMC-Pf at various forces, the volume, density, solubility and antioxidant activity were evaluated. Data from precompressed CMC-Pf (pCMC-Pf) were compared to those of untreated (CMC-Pf) samples

### 6.1 Materials

Peschiera fuchsiaefolia (Pf) extract was purchased from Yerbalatina Phytoactives (Colombo, PR, Brazil) and Artemisinin from Changsha Inner Natural Inc., (Changsha, Hunan, China). Sodium carboxymethylcellulose high viscosity (CMC) was provided by Galenova (St-Hyacinthe, QC, Canada). The ABTS (2,2-azino-bis (3-ethylbenzothiazoline-6-sulfonic acid)) diammonium salt (purity 98%), and Trolox (6-hydroxy-2,5,7,8-tetramethyl-chroman-2-carboxylic acid) 97% were from Sigma-Aldrich (St. Louis, MO, USA). The hydroxypropylmethylcellulose (HPMC) was purchased from The Dow Chemical Company (Midland, MI, USA).

All other reagents used were of analytical grade and used without further purification.

### 6.2 Preparation of CMC-Pf Complex

The CMC/Pf complex powders were obtained by spray-drying (Büchi Mini Spray Dryer B-290, Büchi Labortechnik AG, Flawil, Switzerland). Practically, CMC with a substitution degree about 80%, was dispersed at 1% in water. After homogenization, 400 mL of CMC suspension was slowly added in 480 ml of Pf 20% dissolved in ethanol 60%. This solution was maintained under agitation at room temperature until spray-drying to obtain CMC-Pf complex powder. During the drying process, the Pf/CMC suspension was continuously gentle stirred avoiding sedimentation. The spray-dry parameters were: inlet temperature 150° C., outlet temperature 100° C., spray flow 500 L/h and aspirator setting at 38 m³/h.

### 6.3 Precompression of CMC-Pf Powder

Precompression consisted to exert various forces on the samples. For instance, an amount of CMC-Pf sample powder (20 g) was put in a resistant cotton bag (70 mm diameter and 40 cm length), closed and submitted to initial precompression at 0.5-2.0 tons. Precompressed CMC-Pf powder forms were retained and the volumes and masses of powders were determined for each applied force. According to the reduction of the powder volume, the sample of interest was selected for further use and hereto called precompressed CMC-Pf (pCMC-Pf) complex.

### 6.4 Determination of Tapped Volume: Tap Density Assay

Initial and tapped volume (mL) of CMC-Pf and of pCMC-Pf powders were measured before and after tap density (TD) assay, through a transparent graduated cylinder. The tap density assay was realized by cylinder method (Aulton et al., 1988, using the Vankel tap density apparatus (Varian, Inc., Cary, NC, USA). Tap density assay were carried out with a fixed mass (3 g) of CMC-Pf or pCMC-Pf during 30 to 210 seconds until obtaining a constant volume. The tap density (g/mL) was calculated as following:

$$TD = m / {V}_{T}$$

Where m is the mass of powder and V_{T} is the tapped volume.
   6.5 Characterization of CMC-Pf and pCMC-Pf Complexes

### 6.5.1 Fourier Transform Infrared (FTIR) Spectroscopy

The FTIR spectra of CMC-Pf complex were acquired before and after pre-compaction (as pCMC-Pf). The spectra of CMC-Pf and of pCMC-Pf complexes were obtained on Thermo Scientific Nicolet 6700/Smart iTR (Madison, WI, USA) FTIR spectrometer equipped with a deuterated triglycinesulfate-KBr (DTGS-KBr) detector and a diamond smart attenuated total reflection platform. The crystal was cleaned with ethanol and a background spectrum was acquired, before all assay. All analyses were carried out at room temperature (25±1° C.) using as parameters: number of scans, 32; resolution, 4 cm⁻¹.

### 6.5.2 X-Ray Diffraction (X-RD)

The diffraction patterns of CMC-Pf and pCMC-Pf complexes were recorded using a Siemens D-5000 diffractometer (Munich, Germany) with a SOL-X detector and a Cobalt cathode in reflectance mode at a wavelength of 1.789 Å. The diffractograms were registered over an angular range of 2θ from 0° to 30° at a scan rate of 2° per minute and treated using Diffracplus software.

### 6.5.3 Scanning Electron Microscopy (SEM)

Micrographs of the microstructures of CMC-Pf pCMC-Pf complexes were taken with a JSM-6010LV InTouchScope™ (SEM) (JEOL, Tokyo, Japan) using a secondary electron image detector. A small amount of powder was placed on a metal stub with an adhesive and covered with a fine layer of gold in a sputter coater BIO-RAD E5200 (Bio-Rad Laboratories Ltd., London, UK) under vacuum. The micrographs were obtained by accelerating voltages of 1.5 kV and high vacuum.

### 6.5.4 Thermogravimetry Analysis (TGA)

Thermogravimetric analyses were performed with a TA Instruments TGA (Q500)/Discovery MS. Samples of CMC-Pf and pCMC-Pf (1-10 mg), placed in Pt pans, were heated from 30 to 1000° C. at a temperature variation rate of 10° C./min, under a constant nitrogen flow (100 mL/min). Then, thermogravimetric curves were obtained and the weight loss (%), the onset and the end of sample deterioration were determined.

### 6.6 Solubility Assay

Solubility of CMC-Pf and pCMC-Pf were evaluated according to Konan et al., (2018a) approach, in different solvents: nanopure water, Simulated Gastric Fluid (SGF) or Simulated Intestinal Fluid (SIF). This assay is based on a distinctive property of the tested sample to determine solubility value (mg/mL); it corresponds to the maxima concentration (mg/mL) of sample in a solvent after saturation. Excess of CMC-Pf or pCMC-Pf complexes (2 g) was added carefully in 10 mL of the solvent. The obtained saturated solution was shaken at 37° C. for 5 h and the mixture was centrifuged at 2000×g for 10 min. The supernatant was filtered with 0.45 μm Millipore filter. Then, the effective concentration (mg/mL) of Pf in an aliquot (1 mL) was evaluated by absorbance at 280 nm using the Pf absorbance standard curve at 280 nm as a function of sample concentration (mg/mL).

### 6.7 Antioxidant Properties

The antioxidant activity of CMC-Pf was evaluated by the improved TEAC (Trolox Equivalent Antioxidant Capacity) rapid assay as described by Konan et al., 2016 before and after precompression process. The samples were dissolved in solvent (nanopure water, SGF or SIF). To generate the ABTS•+ radical probe, a volume of 6.5 mL (140 μM in 0.85% NaCl) ABTS was electrolyzed (400 Volts, 10 mA) during 20 seconds. The two electrodes were in platinum, with 2.12 mm diameter, 25.33 mm length and inter-electrode space of 25 mm.

For determination of antioxidant capacity, the ABTS^{•+} solution (950 μL) was mixed with 50 μL of 0-200 mM Trolox (positive control and reference) or 50 μL of the tested samples (Pf, MD/Pf or CMC-Pf in each assay medium) and the absorbance at 734 nm was measured after 1 min of the initial mixture with the electrolysis-induced ABTS^{•+}. The decrease of absorbency due to the antioxidant capacity reflects the scavenging of the free radical (ABTS^{•+}). The ability of Pf, MD/Pf or of CMC-Pf to scavenge the ABTS^{•+} radical cations was compared to that of Trolox (structural hydrosoluble analogue of vitamin E) used as reference antioxidant. The Trolox equivalence corresponds to the Trolox concentration having the same activity as the tested sample at a given concentration. The results are expressed in mM of Trolox equivalence per mass (mg) of sample.

Each measurement consisted of at least five readings and the values represented the mean±standard deviations.

### 6.8 Formulation of Monolithic Dosage Forms and Dissolution Assay

An antimarial Artemisinin-based Combination Therapy (ACT) tablet as previously formulated with Artemisinin and using CMC-Pf complex (Konan et al., 2018b) was also obtained with pCMC-Pf. These monolithic tablet dosage forms contained 100 mg of Arte, 50 mg of croscarmellose cellulose (XC), 100 mg of HPMC, 200 mg of CMC, 15 mg of magnesium stearate and 400 mg of CMC-Pf or pCMC-Pf. Each formulation was directly compacted in the flat-faced punches to obtain monolithic tablets.

Dissolution assays was followed for each tablet first in simulated gastric fluid (SGF, pH 1.5) for 2 h and then continued in simulated intestinal fluid (SIF, pH 6.8) as referred by USP method 40 (USP, 2017c). Release kinetics from both formulation types (containing CMP-Pf or pCMC-Pf) were followed using a Distek dissolution system 2100A (Betatek Inc., Markham, ON, Canada) with a Distek TCS 0200 (North Brunswick, NJ, USA) at 100 rpm and 37° C. At predetermined intervals (0.5, 1, 2, 4, 6, 8, 10 and 12 h), released Pf and Arte from tablets were quantified by absorbancy at 280 nm (for Pf) and at 734 nm (for Arte, using ABTS•+ radical as probe reagent). Practically, for Pf dosage, an established volume (5 mL) was withdrawn from dissolution medium, properly filtered (0.45 um) and the absorbance was measured at 280 nm. The released Arte was recuperated, washed to eliminate Pf residue and then quantified for the release profile by spectrophotometric method using ABTS reagent (Konan et al., 2018b).

### 6.9 Mechanic Properties of the Tablets

### 6.9.1 Friability Test

Tablets friability was determined according to USP/1216 using a tablet friability tester (Varian, Inc., Cary, NC, USA). Ten (10) tablets weighed together, were placed in the friabilator drum and subjected to 100 revolutions at 25 rpm. Then, tablets were cleaned (dust-free), reweighed and their relative weight loss percentage (% Friability) was calculated as:

$$\% F = ( 1 - W / {W}_{o} ) \times 100$$

Where F is friability, Wₒ is the weight of tablets before and W is the weight of tablets after the test.

### 6.9.1.2 Hardness Assay

The hardness test was realized according to USP/1217. The Tensile strength (T) of tablets was:

$$T = 2 F / π \times d \times t$$

F, d and t denotes crushing strength, diameter, and thickness of tablet, respectively. The crushing strength F (the force required to break a tablet in a diametric compression was measured using a VK 200 tablet hardness tester (Varian, Inc., Cary, NC, USA). Diameter and thickness were assessed using an Electronic Digital Caliper (Docap Corp., St-Laurent, Quebec, Canada) for the range 0-200 mm with readings at a precision of tens of microns. F was expressed in kilopond Kp, equivalent to Kg force or 9.81 Newton (N).

Tablets were oval, so the crushing strength was applied to the tablets by taking the length (l) or width (w) of tablet as diameter. In each case with both diameters (diameter as length dl and width dw), five tablets were used. The force required to break the tablet by a diametric compression was recorded.

### 6.10 Statistical Analysis

All experiments were realized at least in triplicate and statistical significance was calculated by one-way ANOVA. Data were presented as mean±standard deviation.

### 6.11 Results and Discussion

In order to reduce the density and volume occupied by Peschiera fuchsiaefolia (under complexed CMC-Pf form) in ACT tablet formulation, the fluffy powder was submitted to a precompression process.

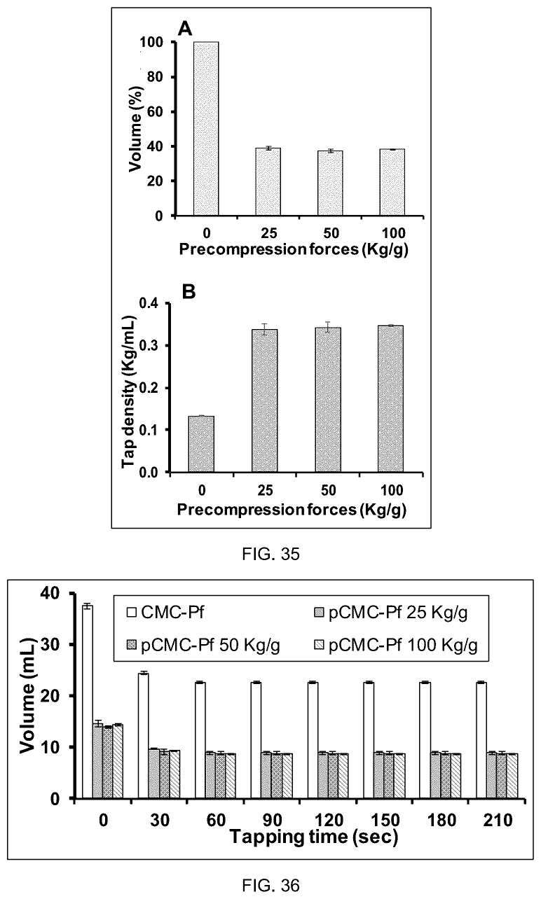

The FIG. 35 presents the initial volumes and volumes of precompressed CMC-Pf (FIG. 35A) and the tap densities (FIG. 35B). Volume of CMC-Pf powder was markedly reduced when it was precompressed at 25, 50 or 100 Kg/g (FIG. 35A). A reduction of about 60% volume of CMC-Pf powder was noticed with forces from 25 Kg/g and no additional improvement was found with higher forces. FIG. 36 shows volumes of CMC-Pf precompressed at different forces, at various tapping time with the effect of tapping on the volume of CMC-Pf and precompressed CMC-Pf. This reduction was found even at short tapping time (30 sec) with no further decrease of volume at longer tapping times up to 210 sec.

Tap density (TD) assay permitted to measure volume (FIG. 36) without interparticulate or intergranulate air. During tapping, the ratio of precompressed CMC-Pf volume to CMC-Pf was approximatively one-third. Practically, precompression would reduce air in the CMC-Pf powder (more inter and intraparticulate air in CMC-Pf than pCMC-Pf. Constant tapped volumes were obtained from 30 sec of tapping for different pCMC-Pf and after 60 sec for native CMC-Pf (uncompressed). The TD values (FIG. 36) were calculated with constant tapped volume showing that pCMC-Pf were three time denser than untreated CMC-Pf. Thus, for the same mass of powder, the pCMC-Pf occupied one-third of CMC-Pf volume.

It appears that precompression from 25 Kg/g caused a significant reduction of CMC-Pf powder and this process was continued for further formulations.

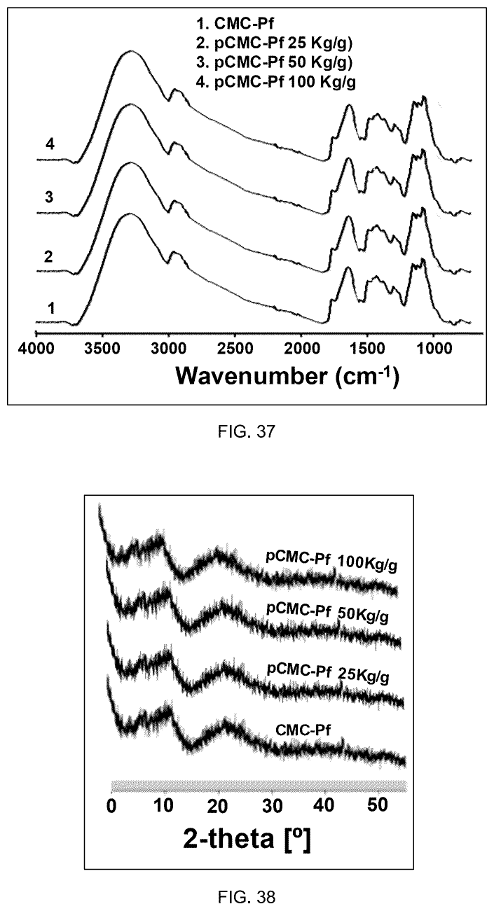

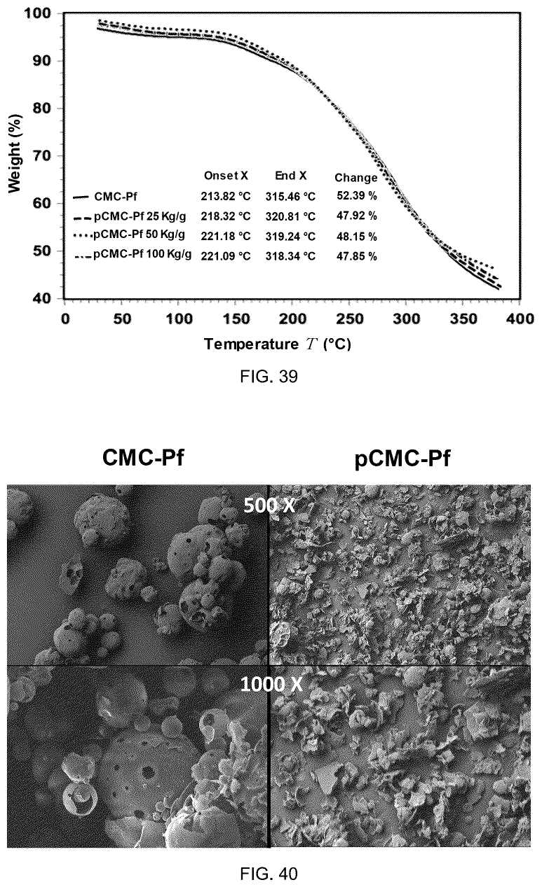

The FTIR spectra (FIG. 37), X-RD diffractograms (FIG. 38) and the TGA diagrams (FIG. 39) confirmed that precompression did not modify the CMC-Pf complex. The characteristic bands (FTIR), the crystallinity (X ray) and the TGA properties (onset, endset, change %) appeared as similar for CMC-Pf and pCMC-Pf. No defection or alteration of the CMC-Pf complexes were found. This is a major advantage for further processing because Pf is more soluble under this complexed form. This form of Pf facilitated the formulation of oral tablet dosage forms by extending the release time and conserved its antioxidant property. Precompression would allow the compaction of CMC-Pf, enhancing the density and improving of the flowability (advantageous for industry) and still maintaining structural characteristics of the complex.

Since no additional improvement was found with precompression forces higher than 25 Kg/g of sample, this value was selected as precompression force and used for further processing.

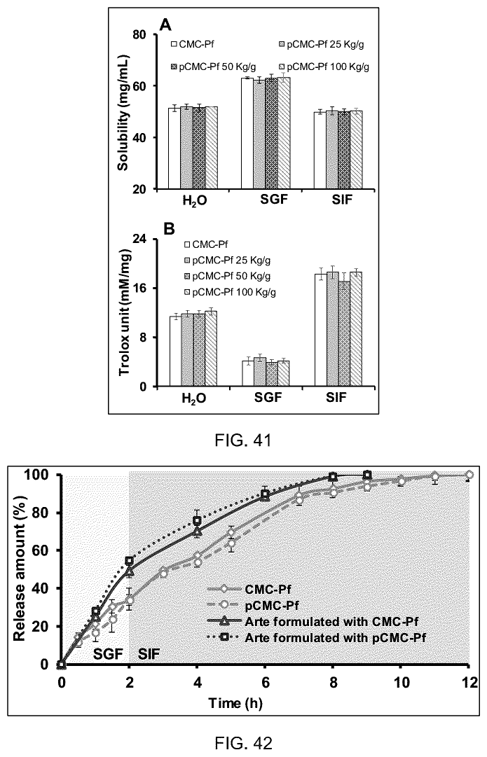

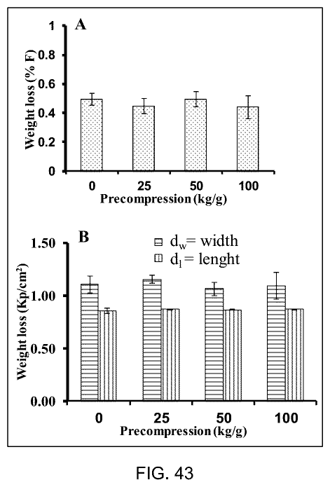

Images from the SEM (FIG. 40) showed that the spheroidal morphology of CMC-Pf was broken by the precompression forces. In fact, the complexation of CMC to Pf via spray drying generated CMC-Pf powder consisting of spheroidal granules containing air resulting in a fluffy and dispersive powder. The precompression produced the breakage of spheroidal shape expelling the intragranular air (FIG. 40), favoring the stacking and pushing together the resulted non-spherical particles with densification. The precompression from 25 Kg/g to 100 Kg/g of CMC-Pf did not affect the solubility of CMC-Pf (FIG. 41A), nor the antioxidant capacity (FIG. 41B), neither the release profiles of CMC-Pf and Arte in the ACT from monolithic tablet formulation (FIG. 42). Also, mechanical characteristics were preserved after precompression (FIG. 43).

In the two formulations (FIG. 43), Arte was released about 51% after 2 h in SGF and the complete release was achieved after 8 h. Differently, Pf associated to CMC as CMC-Pf and the pCMC-Pf was slowly released during 10-12 h. Practically, no significant changes in terms of release patterns were observed for uncompressed CMC-Pf and Arte versus precompressed Pf and Arte released from the monolithic dosage forms. Furthermore, the size, shape and weight of the tablets remained almost unchanged using uncompressed or precompressed bioactive CMC-Pf complexes. The mechanical properties were also maintained (FIG. 43A) with a weight loss in the frame of the tolerated limit 1% (Podczeck, 2012). The tensile strengths were also maintained after precompression (FIG. 43B). Thereby, tablets would resist to crushing, capping, fracture, cracking, cleavage, breakage or abrasion before use. They would withstand to the stress of packaging, transportation and handling.

### 6.12 Conclusion

Precompression from 25 Kg per g of CMC-Pf powder favored a marked reduction of the powder volume. It permitted to conserve the chemical (functional groups, structure), biological (antioxidant activity) and pharmacokinetic (solubility, release kinetic profile) characteristics of tablets. Only the morphology of particles was affected. The precompression at 25 Kg/g was appropriate to resolve the problem related to the air trapped in the Pf granules and to improve the compactibility and flowability of Pf/CMC powder. This precompression method is low cost, easy to manufacture and particularly, no extra ingredients added in the formulation. Furthermore, it will facilitate the use of CMC-Pf complexes in antimalarial formulation. Also, it is simple, rapid and not expensive to realize with no highly sophisticated equipment.

While preferred embodiments have been described above and illustrated in the accompanying drawings, it will be evident to those skilled in the art that modifications may be made without departing from this disclosure. Such modifications are considered as possible variants comprised in the scope of the disclosure.

### REFERENCES

1. Ancolio C., Azas N., Mahiou V., Ollivier E., Di Giorgio C., Keita A., Timon-David P., Balansard G., 2002. Antimalarial activity of extracts and alkaloids isolated from six plants used in traditional medicine in Mali and Sao Tome. Phytother Res., 16, 646-649.
2. Asrade S., Mengesha Y., Moges G., Gelayee D.A., 2017. In vivo antiplasmodial activity evaluation of the leaves of Balanites rotundifolia (Van Tiegh.) Blatter (Balanitaceae) against Plasmodium berghei. J.Exp. Pharmacol., 9, 59-66.
3. Assaad E, Mateescu MA., 2010. The influence of protonation ratio on properties of carboxymethyl starch excipient at various substitution degrees: Structural insights and drug release kinetics. Int J Pharm., 15, 75-84.
4. Cano-Chauca M., Stringheta PC., Ramos AM., Cal-Vidal J., 2005. Effect of carriers on the microstructure of mango powder obtained by spray drying and its functional characterization. Innov Food Sci Emerg Tech., 6, 420-428.
5. Canuto S., Fileti E.E., Chaudhuri P., 2004. Relative strength of hydrogen bond interaction in alcohol-water complexes. Chem Phys Lett, 400, 494-499.
6. Chowdhury S.R., Kumar A., Godinho J.L.P, De Macedo S.T., Zuma A.A; Sourav S., Kumari N., Rodrigues J.C.F, Sundard S., Dujardin J.C., Roy S., De Souza W., Mukhopadhyay S., Majumdera K.H., 2017. Voacamine alters Leishmania ultrastructure and kills parasite by poisoning unusual bi-subunit topoisomerase IB. Biochem. Pharmacol., 138, 19-30.
7. CCD, 2017. Combined Chemical Dictionary. Consulted 2017 Nov. 2^{nd}, under all text: Alkaloid: http://ccd.chemnetbase.com/faces/chemical/ChemicalSearchResults.xhtml
8. Konan K.V., Le Tien C. and Mateescu M.A., 2016. Electrolysis-induced fast activation of the ABTS reagent for an antioxidant capacity assay. Anal. Methods, 8, 5638-5644.
9. Le Tien C., Millette M., Mateescu M.A, Lacroix M., 2004. Modified alginate and chitosan for lactic acid bacteria immobilization. Biotechnol Appl Biochem., 39:347-54.
10. Mohammadi G., Nokhodchi A., Barzegar-Jalali M., Lotfipour F., Adibkia K., Ehyaei N., Valizadeh H., 2011. Physicochemical and anti-bacterial performance characterization of clarithromycin nanoparticles as colloidal drug delivery system. Colloids Surf. B, 88, 39-44.
11. Munoz V., Moretti C., Sauvain M., Caron C., Porzel A., Massiot G., Richard b., Le Men-Olivier L., 1994. Isolation of bis-indole alkaloids with antileishmanial and antibacterial activities from Peschiera van heurkii (syn. Tabernaemontana van heurkii) Planta. Med., 60, 455-459.
12. Nair J.J., Wilhelm A., Bonnet S.L., Stade J.V., 2017. Antibacterial constituents of the plant family Amaryllidaceae. Bioorg. Med. Chem. Lett., 27.4943-4951.

13. Stojanovic Zeljko, Jeremic Katarina, Jovanovic S. and Lechner M.D., 2005. A Comparison of Some Methods for the Determination of the Degree of Substitution of Carboxymethyl Starch. Starch/Stärke, 57, 79-83.

14. USP, 2017a. U.S Pharmacopoeia-National Formulary (USP 40 NF 35). Vol 1. Rockville, Md, USA: United States Pharmacopeial Convention, Inc; 2016. Description and Relative solubility of USP and NF Articles; p. 2453-2454.
15. USP, 2017b. U.S Pharmacopoeia-National Formulary (USP 40 NF 35). Vol 1. Rockville, Md, USA: United States Pharmacopeial Convention, Inc; 2016. Dissolution; p. 588-610.
16. Haynes, R.K., Hung-On Pai, H., Voerste, A. 1999. Ring opening of artemisinin (qinghaosu) and dihydroartemisinin and interception of the open hydroperoxides with Formation of N-oxides—a chemical model for antimalarial mode of action. Tetrahedron Lett., 40, 4715-4718.
17. Li X., Gao W-Y, Huang L-J, Wang Y-L, Huang L-Q, Liu C-X., 2010. Preparation and physicochemical properties of carboxymethyl Fritillaria ussuriensis Maxim. starches. Carbohydr Polym., 47, 768-773.
18. Podczeck F., 2012. Methods for the practical determination of the mechanical strength of tablets-From empiricism to science. Int. J. Pharm., 436, 214-232.
19. Ramanitrahasimbola D, Rasoanaivo P, Ratsimamanga-Urverg S, Federici E, Palazzino G, Galeffi C et Nicoletti M, 2001. Biological Activities of the Plant derived Bisindole voacamin with Reference to Malaria. Phytother. Res., 15, 30-33.
20. USP, 2017. U.S Pharmacopoeia-National Formulary (USP 40 NF 35). Vol 1. Rockville, Md, USA: United States Pharmacopeial Convention, Inc; 2016. Description and Relative solubility of USP and NF Articles; p. 2453-2454.
21. Zhang B., Tao H., Wei B., Jin Z., Xu X., Tian Y., 2014. Characterization of different substituted carboxymethylstarch microgels and their interactions with lysozyme. PLOS One, 9, e114634.

1. A controlled release complex comprising:
a carboxylated polymer having carboxyl groups having a degree of substitution of about 0.1 to about 1.0, forming a complex through ionic interaction with a first antimalarial alkaloid extract from Peschiera fuchsiaefolia, wherein said carboxylated polymer and said antimalarial alkaloid extract are present in a ratio of from about 10:90 to about 1:99.

2. The controlled release complex of claim 1, wherein said carboxylated polymer is selected from the group consisting of carboxymethylcellulose (CMC), carboxymethylstarch (CMS), or combinations thereof.

3. The controlled release complex of claim 1, wherein said carboxymethylcellulose has a degree of substitution of about 0.7 to about 0.8.

4. The controlled release complex of claim 1, wherein said carboxymethylcellulose has a degree of substitution of about 0.8.

5. The controlled release complex of claim 1, wherein said carboxymethylstarch has a degree of substitution of about 0.1 to about 0.3.

6. The controlled release complex of claim 1, wherein said carboxymethylstarch has a degree of substitution of about 0.27.

7. The controlled release complex of claim 1, wherein said carboxylated polymer and said antimalarial alkaloid extract are present in a ratio of about 4:96.

8. The controlled release complex of claim 1, wherein said antimalarial alkaloid extract further comprises a second antimalarial alkaloid extract selected from the group consisting of an alkaloid extract from Guiera senegalensis, Strychnos usambarensis, and Balanites rotundifolia.

9. A solid oral dosage form comprising the controlled release complex of claim 1 and pharmaceutically acceptable excipients.

10. The solid oral dosage form of claim 9, further comprising any one of:
a) an antimalarial drug;
b) a binding agent;
c) a lubricant; and
d) an additional carboxylated polymer, uncomplexed with said antimalarial alkaloid extract.

11. The solid oral dosage form of claim 10, wherein said binding agent is selected from the group consisting of hydroxypropyl methylcellulose (HPMC), sucrose, lactose, starches, cellulose, microcrystalline cellulose, xylitol, sorbitol, mannitol, gelatin, hydroxypropyl cellulose (HPC), polyvinylpyrrolidone (PVP), and polyethylene glycol (PEG).

12. The solid oral dosage form of claim 10, wherein said lubricant is selected from the group consisting of magnesium stearate, talc, silica, vegetable stearin, and stearic acid.

13. The solid oral dosage form of claim 9, wherein said dosage form is monolithic or multilayered.

14. The solid oral dosage form of claim 9, where said antimalarial drug is selected from the group consisting of quinine, artemisinin, artesunate, artemether, arteether, dihydroartemisinin, and artelinate.

15. The solid oral dosage form of claim 13, where said antimalarial drug is artemisinin.

16. The solid oral dosage form of claim 13, wherein said solid oral dosage form is a bilayer.

17. A method of treating malaria comprising administering to a subject in need thereof an effective amount of the solid dosage form of claim 9.

18. A method of treating malaria comprising administering to a subject in need thereof an effective amount of the solid dosage form of claim 10.
